SEQUENCE LISTING (34 sequences)

SEQ ID NO: 1 - protein, 118 aa, synthetic construct
DVQLQESGPDLVKPSQSLSLTCTVTGYSITSGYTWHWIRQFPGNTVEWMGYIQYTGSTRYNPALRGRLSISRDTSKNQFFLQLISVTTADTGTYFCARGTTASFDYWGQGTTLTVASA

SEQ ID NO: 2 - protein, 117 aa, synthetic construct
DVQLQESGPGLVKPSQTLSLTCTVTGYSITSGYTWHWIRQPPGKGLEWMGYIQYTGSTRYNPSLKSRVTISRDTSKNQFSLKLSSVTAADTAVYFCARGTTASFDYWGQGTTVTVSS

SEQ ID NO: 3 - protein, 117 aa, synthetic construct; identical to SEQ ID NO: 2

SEQ ID NO: 4 - protein, 117 aa, synthetic construct; identical to SEQ ID NO: 2

SEQ ID NO: 5 - protein, 117 aa, synthetic construct
QVQLQESGPGLVKPSQTLSLTCTVTGYSITSGYTWSWIRQPPGKGLEWIGYIQYTGSTRYNPSLKSRVTISRDTSKNQFSLKLSSVTAADTAVYYCARGTTASFDYWGQGTTVTVSS

SEQ ID NO: 6 - protein, 117 aa, synthetic construct; identical to SEQ ID NO: 5

SEQ ID NO: 7 - protein, 117 aa, synthetic construct; identical to SEQ ID NO: 5

SEQ ID NO: 8 - protein, 117 aa, synthetic construct
DVQLKESGPALVKPTQTLTLTCTVTGYSITSGYTWHWIRQPPGKALEWMGYIQYTGSTRYNPSLKTRLTISRDTSKNQFVLTMTNMDPVDTATYFCARGTTASFDYWGQGTTVTVSS

SEQ ID NO: 9 - protein, 117 aa, synthetic construct; identical to SEQ ID NO: 8

SEQ ID NO: 10 - protein, 117 aa, synthetic construct
QVTLKESGPALVKPTQTLTLTCTVTGYSITSGYTVSWIRQPPGKALEWIGYIQYTGSTRYNPSLKTRLTISRDTSKNQFVLTMTNMDPVDTATYYCARGTTASFDYWGQGTTVTVSS

SEQ ID NO: 11 - protein, 117 aa, synthetic construct; identical to SEQ ID NO: 10

SEQ ID NO: 12 - protein, 117 aa, synthetic construct; identical to SEQ ID NO: 10

SEQ ID NO: 13 - protein, 117 aa, synthetic construct
QVQLQESGPGLVKPSQTLSLTCTVTGYSITSGYTWSWIRQHPGKGLEWMGYIQYTGSTRYNPSLKSLLTISRDTSKNQFSLKLSSVTAADTAVYYCARGTTASFDYWGQGTTVTVSS

SEQ ID NO: 14 - protein, 117 aa, synthetic construct
QVQLQESGPGLVKPSQTLSLTCTVTGYSITSGYTWSWIRQHPGKGLEWMGYIQYTGSTRYNPSLKSLVTISRDTSKNQFSLKLSSVTAADTAVYYCARGTTASFDYWGQGTTVTVSS

SEQ ID NO: 15 - protein, 447 aa, synthetic construct
DVQLQESGPDLVKPSQSLSLTCTVTGYSITSGYTWHWIRQFPGNTVEWMGYIQYTGSTRYNPALRGRLSISRDTSKNQFFLQLISVTTADTGTYFCARGTTASFDYWGQGTTLTVASAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 16 - protein, 447 aa, synthetic construct
DVQLQESGPDLVKPSQSLSLTCTVTGYSITSGYTWHWIRQFPGNTVEWMGYIQYTGSTRYNPALRGRLSISRDTSKNQFFLQLISVTTADTGTYFCARGTTASFDYWGQGTTLTVASAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 17 - protein, 447 aa, synthetic construct
DVQLQESGPDLVKPSQSLSLTCTVTGYSITSGYTWHWIRQFPGNTVEWMGYIQYTGSTRYNPALRGRLSISRDTSKNQFFLQLISVTTADTGTYFCARGTTASFDYWGQGTTLTVASAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFACAVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 18 - protein, 113 aa, synthetic construct
DIVMSQSPSSLNVSVGEKVTMRCRSSQSLLYSSDQKNYLTWYQQKPGQSPKLLISWASTRASGVPDRFTGSGSGTDFTLTISSVKAEDLGVYYCQQYFIYPLTFGVGTKLGLK

SEQ ID NO: 19 - protein, 113 aa, synthetic construct
DIVMTQSPLSLPVTPGEPASISCRSSQSLLYSSDQKNYLTWYLQKPGQSPQLLISWASTRASGVPDRFSGSGSGTDFTLKISRVEAEDVGVYYCQQYFIYPLTFGGGTKLEIK

SEQ ID NO: 20 - protein, 113 aa, synthetic construct
DIQMTQSPSSLSASVGDRVTITCRSSQSLLYSSDQKNYLTWYQQKPGKAPKLLISWASTRASGVPSRFSGSGSGTDFTLTISSLQPEDFATYYCQQYFIYPLTFGGGTKLEIK

SEQ ID NO: 21 - protein, 113 aa, synthetic construct
DIQMTQSPSSLSASVGDRVTITCRSSQSLLYSSDQKNYLTWYQQKPGKAPKLLISWASTLASGVPSRFSGSGSGTDFTLTISSLQPEDFATYYCQQYFIYPLTFGGGTKLEIK

SEQ ID NO: 22 - protein, 113 aa, synthetic construct; identical to SEQ ID NO: 19

SEQ ID NO: 23 - protein, 113 aa, synthetic construct; identical to SEQ ID NO: 20

SEQ ID NO: 24 - protein, 113 aa, synthetic construct; identical to SEQ ID NO: 21

SEQ ID NO: 25 - protein, 113 aa, synthetic construct
DIVMTQSPDSLAVSLGERATINCKSSQSLLYSSDQKNYLTWYQQKPGQPPKLLISWASTRASGVPDRFSGSGSGTDFTLTISSLQAEDVAVYYCQQYFIYPLTFGGGTKLEIK

SEQ ID NO: 26 - protein, 113 aa, synthetic construct; identical to SEQ ID NO: 19

SEQ ID NO: 27 - protein, 113 aa, synthetic construct; identical to SEQ ID NO: 19

SEQ ID NO: 28 - protein, 113 aa, synthetic construct; identical to SEQ ID NO: 20

SEQ ID NO: 29 - protein, 113 aa, synthetic construct; identical to SEQ ID NO: 21

SEQ ID NO: 30 - protein, 113 aa, synthetic construct
DIVMTQSPDSLAVSLGERATINCKSSQSLLYSSDQKNYLAWYQQKPGQSPKLLIYWASTRESGVPDRFSGSGSGTDFTLTISSLQAEDVAVYYCQQYFIYPLTFGQGTKLEIK

SEQ ID NO: 31 - protein, 113 aa, synthetic construct; identical to SEQ ID NO: 30

SEQ ID NO: 32 - protein, 220 aa, synthetic construct
DIVMSQSPSSLNVSVGEKVTMRCRSSQSLLYSSDQKNYLTWYQQKPGQSPKLLISWASTRASGVPDRFTGSGSGTDFTLTISSVKAEDLGVYYCQQYFIYPLTFGVGTKLGLKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 33 - protein, 220 aa, synthetic construct; identical to SEQ ID NO: 32

SEQ ID NO: 34 - protein, 220 aa, synthetic construct; identical to SEQ ID NO: 32

(19) United States
(12) Patent Application Publication
(10) Pub. No.: US 2025/0304711 A1
(43) Pub. Date: Oct. 2, 2025

(54) Methods and Compositions for Treating Obesity, Obesity-Related Diseases, Fatty Liver and Fatty Liver Associated Diseases with Anti-CD24 Agents

(71) Applicant: The Board of Trustees of the Leland Stanford Junior University, Stanford, CA (US)

(72) Inventors: Jeffrey S. Glenn, Stanford, CA (US); Edward A. Pham, Stanford, CA (US); Matthew F. Yee, Stanford, CA (US); Da Yoon No, Stanford, CA (US)

(21) Appl. No.: 18/862,297

(22) PCT Filed: May 19, 2023
(86) PCT No.: PCT/US2023/067246

§ 371 Date: Nov. 1, 2024

Related U.S. Application Data
(60) Provisional application No. 63/344,410, filed on May 20, 2022.

(51) Int. Cl. C07K 16/28; A61K 31/4184; A61K 31/4192; A61K 39/00; A61P 1/16

(52) U.S. Cl. CPC C07K 16/2896; A61K 31/4184; A61K 31/4192; A61P 1/16; A61K 2039/505

(57) ABSTRACT

Methods and compositions for treating obesity, obesity-related diseases, fatty liver and fatty liver associated diseases are provided. Aspects of the methods include administering to a subject in need thereof an effective amount of an anti-CD24 agent to treat the subject. Also provided are compositions for use in practicing embodiments of the methods.

### CROSS-REFERENCE

This application claims the benefit of U.S. Provisional Patent Application No. 63/344,410, filed May 20, 2022, which application is incorporated herein by reference in its entirety.

Incorporation By Reference Of Sequence Listing Provided As A Sequence Listing Xml File A Sequence Listing is provided herewith as a Sequence Listing XML, “STAN-1959WO_SEQ_LIST.xml” created on May 15, 2023 and having a size of 35,582 bytes. The contents of the Sequence Listing XML are incorporated by reference herein in their entirety.

### INTRODUCTION

Obesity and obesity-associated diseases are major causes of morbidity and mortality world-wide. One major obesity-associated disease is fatty liver disease. In addition to obesity, fatty liver disease can be from other causes. For example, it can be a result of excessive alcohol assumption, genetic predisposition, viral hepatitis, exposure to chemicals such as perfluorooctanoic acid (PFA), and metabolic syndrome. It is estimated that 1 billion people world-wide, and about one third of the US population, or 100 million American have nonalcoholic fatty liver diseases (NAFLD). In many patients, NAFLD can progress to non-alcoholic steatohepatitis (NASH) that can ultimately result in liver failure and/or liver cancer.

In addition, many patients with end-stage liver diseases (ESLD) require liver transplantation as a life-saving therapy. The presence of significant steatosis in the donor liver can increase the risk of graft failure and can lead to increased mortalities and morbidities in liver transplant patients.

### SUMMARY

Methods and compositions for treating obesity, obesity-related diseases, fatty liver and fatty liver associated diseases are provided. Aspects of the methods include administering to a subject in need thereof an effective amount of an anti-CD24 agent to treat the subject. Methods and compositions for reducing steatosis (hepatic steatosis) are provided. Aspects of the methods include administering to a subject in need thereof an effective amount of an anti-CD24 agent to reduce steatosis (hepatic steatosis). Also provided are compositions for use in practicing embodiments of the methods.

The inventors discovered that anti-CD24 agents reduce steatosis (hepatic steatosis), and therefore can be used to treat a number of ailments/diseases (e.g., fatty liver and obesity related diseases). The inventors demonstrated that a number of different anti-CD24 antibodies, including those with a fully functional Fc region, a “dead” Fc region, or no Fc region at all (e.g., the G7s scFv) can be used to reduce steatosis. Thus, any binding agent that targets/binds to CD24 should function to reduce steatosis. To this end, the inventors demonstrated (see working examples below) that known small molecules, screened for their ability to bind CD24, do in fact reduce steatosis and can therefore be used to treat disease (e.g., fatty liver and obesity related diseases). The inventors realized that any anti-CD24 agent is useful for reducing steatosis/useful for treatment, i.e., treatment is not limited to certain anti-CD24 antibodies, or to anti-CD24 antibodies at all-instead any anti-CD24 agent, including any CD24 binding agent will function to reduce steatosis. For example, the inventors realized that agents that reduce CD24 expression (also referred to herein as anti-CD24 agents) such as RNAi agents (shRNA/siRNA), antisense agents (e.g., antisense oligos, LNAs, and the like), CRISPR agents (e.g., CRISPR nuclease/cleavage activity, CRISPRi, and the like) will also be active for reducing steatosis.

Provided are methods of reducing steatosis in an individual, where the method includes administering to an individual who has steatosis, a therapeutically effective dose of an anti-CD24 agent for a period of time sufficient to reduce the steatosis. In some cases, the subject has non-alcoholic fatty liver disease (NAFLD) or non-alcoholic steatohepatitis (NASH).

In some embodiments, the anti-CD24 agent is an anti-CD24 antibody. In some cases, the anti-CD24 antibody includes an Fc with one or more silencing mutations. In some cases, the anti-CD24 antibody is an anti-CD24 fragment antigen-binding region (Fab) or an anti-CD24 pegylated Fab fragment. In some cases, the anti-CD24 antibody is an anti-CD24 single-chain variable fragment (scFv). In some cases, the anti-CD24 antibody includes the 3 heavy chain and 3 light chain CDRs of SWA11, ML5, SN3, or G7s. In some cases, the anti-CD24 antibody is a humanized antibody. In some cases, the anti-CD24 antibody comprises an amino acid sequence having the 3 heavy chain IMGT CDRs of SWA11 and having 80% or more sequence identity with the humanized variable heavy chain sequence of any one of SEQ ID NOs: 2-14; and an amino acid sequence having the 3 light chain IMGT CDRs of SWA11 and having 80% or more sequence identity with the humanized variable light chain sequence of any one of SEQ ID NOs: 19-31. In some cases, the anti-CD24 agent is a small molecule that binds to CD24.

In some embodiments, the course of treatment for the individual is in a range of from 1-8 weeks. In some cases, administration of the anti-CD24 agent is combined with another therapeutic modality such as dietary modification and/or weight loss surgery. In some cases, the anti-CD24 agent is co-administered with another therapeutic agent.

In some embodiments, the individual is a liver transplant patient and said administering is to enhance liver graft function and/or prolong liver graft survival. In some embodiments, the individual has fatty liver, a fatty liver associated disease, obesity, or an obesity-related disease

### BRIEF DESCRIPTION OF THE FIGURES

FIG. 1A-1D: Relative lipid accumulation of Huh 7D cells when cultured on fatty medium and treated with various anti-CD24 agents. Agents were added at 1 μg/mL unless otherwise specified as in the Fab. MOSW1, SWA11, ML5, SN3: mouse anti human CD24 antibodies; MOSW2: SWA11 antibody with point mutations to silent complement activity; MOSW3: SWA11 antibody with point mutations to silent complement activity, antibody-dependent cytotoxicity (ADCC) and antibody-dependent cellular phagocytosis (ADCP); Fab: Fab of SWA11 antibody, G7s: scFv against human and mouse CD24; TW2, TW6, V2, V3, V4, V6, V7, V8, V9, V10, V14, V15, V16: different humanized anti-CD24 antibodies; hIgG: human IgG isotype control. FIG. 1A: anti-steatosis activity of SWA-11, Fab, TW2, TW6 and G7S; FIG. 1B: anti-steatosis activity of V2, V3, V4, V6, V7, V8, V9, V10, V14, V15, V16; FIG. 1C: anti-steatosis of SWA11, Fab, ML5, SN3, MOSW1, MOSW2, MOSW3; FIG. 1D: anti-steatosis of 3 small molecules that bind CD24.

FIG. 2A-2B: H&E stains of livers from mice on HFD treated with IgG control (FIG. 2A) vs. anti-CD24 agent (FIG. 2B).

FIG. 3: Liver ultrasound of mice on high fat diet treated with IgG control (top) compared to anti-CD24 agent (bottom).

FIG. 4A-4B: H&E stains of livers from ob/ob mice on HFD treated with IgG control (FIG. 4A) vs. anti-CD24 agent (FIG. 4B) for 1 week.

FIG. 5A-5B: H&E stains of livers from ob/ob mice on HFD then placed back on regular chow diet and treated with IgG control (FIG. 5A) vs. anti-CD24 agent (FIG. 5B) for 1 week.

FIG. 6: Serum liver chemistry in ob/ob mice induced with HFD treated with istotype IgG or anti-CD24 agents.

FIG. 7A-7B: Metabolism of clemizole in ob/ob mice treated with IgG control or anti-CD24 agents.

FIG. 8A-8B: H&E stains of livers from ob/ob mice induced on HFD treated with IgG control (FIG. 8A) vs. ADCC-silent anti-CD24 antibody (FIG. 8B) for 1 week while on regular diet.

FIG. 9A-9C: weekly serum human albumin in mice with humanized liver on HFD for 6 weeks then treated with IgG control of MOSW3 for 3 weeks (FIG. 9A) and corresponding CT scan of the liver at 3 weeks after IgG control treatment (FIG. 9B) or MOSW3 treatment (FIG. 9C).

FIG. 10A-10B: Weights of mice treated with IgG control or anti-CD24 agents.

### DEFINITIONS

In the description that follows, a number of terms conventionally used in the field are utilized. In order to provide a clear and consistent understanding of the specification and claims, and the scope to be given to such terms, the following definitions are provided.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refer to compounds that have the same basic chemical structure as a naturally occurring amino acid, i.e., an .alpha. carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. In some embodiments, the mammal is human.

The term “sample” with respect to a patient encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents; washed; or enrichment for certain cell populations. The definition also includes sample that have been enriched for particular types of molecules, e.g., nucleic acids, polypeptides, etc.

The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, aspirate, and the like. A “biological sample” includes a sample comprising target cells and/or normal control cells, or is suspected of comprising such cells. The definition includes biological fluids derived therefrom (e.g., infected cell, etc.), e.g., a sample comprising polynucleotides and/or polypeptides that is obtained from such cells (e.g., a cell lysate or other cell extract comprising polynucleotides and/or polypeptides). A biological sample comprising an infected cell, etc. from a patient can also include non-infected cells.

The term “diagnosis” is used herein to refer to the identification of a molecular or pathological state, disease or condition.

The term “prognosis” is used herein to refer to the prediction of the likelihood of disease progression (e.g., progression of an infection, etc.), including recurrence, drug resistance, etc.

The term “prediction” is used herein to refer to the act of foretelling or estimating, based on observation, experience, or scientific reasoning. In one example, a physician may predict the likelihood that a patient will survive.

The term “specific binding member” as used herein refers to a member of a specific binding pair (i.e., two molecules, usually two different molecules, where one of the molecules, e.g., a first specific binding member, through non-covalent means specifically binds to the other molecule, e.g., a second specific binding member).

The term “specific binding” refers to a direct association between two molecules, due to, for example, covalent, electrostatic, hydrophobic, and ionic and/or hydrogen-bond interactions, including interactions such as salt bridges and water bridges. A specific binding member describes a member of a pair of molecules which have binding specificity for one another. The members of a specific binding pair may be naturally derived or wholly or partially synthetically produced. One member of the pair of molecules has an area on its surface, or a cavity, which specifically binds to and is therefore complementary to a particular spatial and polar organization of the other member of the pair of molecules. Thus, the members of the pair have the property of binding specifically to each other. Examples of pairs of specific binding members are antigen-antibody, biotin-avidin, hormone-hormone receptor, receptor-ligand, enzyme-substrate. Specific binding members of a binding pair exhibit high affinity and binding specificity for binding with each other. Typically, affinity between the specific binding members of a pair is characterized by a K_{d} (dissociation constant) of 10⁻⁶ M or less, such as 10⁻⁷ M or less, including 10⁻⁸ M or less, e.g., 10⁻⁹ M or less, 10⁻¹⁰ M or less, 10⁻¹¹ M or less, 10⁻¹² M or less, 10⁻¹³ M or less, 10⁻¹⁴ M or less, including 10⁻¹⁵ M or less. “Affinity” refers to the strength of binding, increased binding affinity being correlated with a lower KD.

In an embodiment, affinity is determined by surface plasmon resonance (SPR), e.g., as used by Biacore systems. The affinity of one molecule for another molecule is determined by measuring the binding kinetics of the interaction, e.g., at 25° C. “Affinity” refers to the strength of binding, increased binding affinity being correlated with a lower KD. In an embodiment, affinity is determined by surface plasmon resonance (SPR), e.g., as used by Biacore systems. The affinity of one molecule for another molecule is determined by measuring the binding kinetics of the interaction, e.g., at 25° C.

The methods described herein may include multiple steps. Each step may be performed after a predetermined amount of time has elapsed between steps, as desired. As such, the time between performing each step may be 1 second or more, 10 seconds or more, 30 seconds or more, 60 seconds or more, 5 minutes or more, 10 minutes or more, 60 minutes or more and including 5 hours or more. In certain embodiments, each subsequent step is performed immediately after completion of the previous step. In other embodiments, a step may be performed after an incubation or waiting time after completion of the previous step, e.g., a few minutes to an overnight waiting time.

As used herein, the terms “evaluating”, “determining.” “measuring.” and “assessing,” and “assaying” are used interchangeably and include both quantitative and qualitative determinations.

The term “separating”, as used herein, refers to physical separation of two elements (e.g., by size or affinity, etc.) as well as degradation of one element, leaving the other intact.

### DETAILED DESCRIPTION

Methods and compositions for treating obesity, obesity-related diseases, fatty liver and fatty liver associated diseases are provided. Aspects of the methods include administering to a subject in need thereof an effective amount of an anti-CD24 agent to treat the subject. Also provided are compositions for use in practicing embodiments of the methods.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

Methods and compositions for treating obesity, obesity-related diseases, fatty liver and fatty liver associated diseases are provided. Aspects of the methods include administering to a subject in need thereof an effective amount of an anti-CD24 agent to treat the subject. Also provided are compositions for use in practicing embodiments of the methods.

By “treatment” it is meant that at least an amelioration of one or more symptoms associated with target condition afflicting the subject is achieved, where amelioration is used in a broad sense to refer to at least a reduction in the magnitude of a parameter, e.g., a symptom associated with the target condition being treated. As such, treatment also includes situations where a pathological condition, or at least symptoms associated therewith, are completely inhibited, e.g., prevented from happening, or stopped, e.g., terminated, such that the adult mammal no longer suffers from the target condition, or at least the symptoms that characterize the impairment. In some instances, “treatment”, “treating” and the like refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” may be any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. Treatment may result in a variety of different physical manifestations, e.g., modulation in gene expression, rejuvenation of tissue or organs, etc. Treatment of ongoing disease, where the treatment stabilizes or reduces the undesirable clinical symptoms of the patient, occurs in some embodiments. Such treatment may be performed prior to complete loss of function in the affected tissues. The subject therapy may be administered during the symptomatic stage of the disease, and in some cases after the symptomatic stage of the disease.

A therapeutic treatment is one in which the subject is inflicted prior to administration and a prophylactic treatment is one in which the subject is not inflicted prior to administration. In some embodiments, the subject has an increased likelihood of becoming inflicted or is suspected of being inflicted prior to treatment. In some embodiments, the subject is suspected of having an increased likelihood of becoming inflicted.

Methods described herein may be employed to treat any type of subject in need of treatment. Subject include mammalian species. Mammalian species that may be treated with the present methods include canines and felines; equines; bovines; ovines; etc., and primates, including humans. The subject methods, compositions, and reagents may also be applied to animal models, including small mammals, e.g., murine, lagomorpha, etc., for example, in experimental investigations. The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. In some embodiments, the mammal is human. In some embodiments the individual is not human (e.g., a non-human mammal).

Anti-CD24 agents. As summarized above, aspects of the method include administering to the subject an anti-CD24 agent. Anti-CD24 agents of interest include specific binding members for CD24. Specific binding members exhibit an affinity (Kd) for a target CD24, such as human CD24, that is sufficient to provide for the desired treatment. As used herein, the term “affinity” refers to the equilibrium constant for the reversible binding of two agents; “affinity” can be expressed as a dissociation constant (Kd). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater, or more, than the affinity of an antibody for unrelated amino acid sequences. Affinity of a specific binding member to a target protein can be, for example, from about 100 nanomolar (nM) to about 0.1 nM, from about 100 nM to about 1 picomolar (pM), or from about 100 nM to about 1 femtomolar (fM) or more. The term “binding” refers to a direct association between two molecules, due to, for example, covalent, electrostatic, hydrophobic, and ionic and/or hydrogen-bond interactions, including interactions such as salt bridges and water bridges. In some embodiments, an anti-CD24 agent binds human CD24 with nanomolar affinity or picomolar affinity. In some embodiments, the anti-CD24 agent binds human CD24 with a Kd of less than about 100 nM, 50 nM, 20 nM, 20 nM, or 1 nM. Anti-CD24 agents in some cases are antibodies. In some embodiments, the antibodies bind human CD24 with nanomolar affinity or picomolar affinity. In some embodiments, the antibodies bind human CD24 with a Kd of less than about 100 nM, 50 nM, 20 nM, 20 nM, or 1 nM.

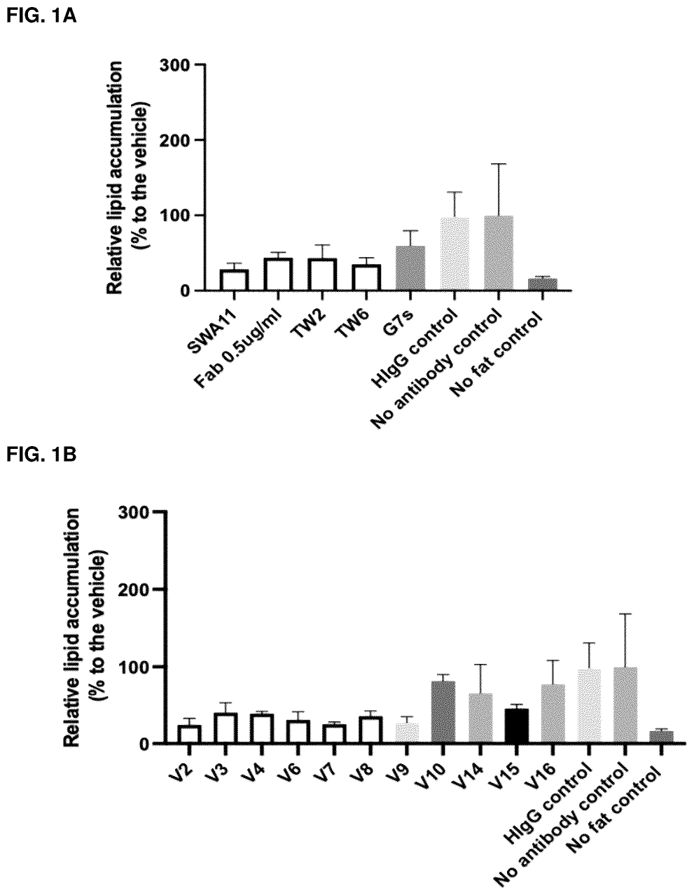

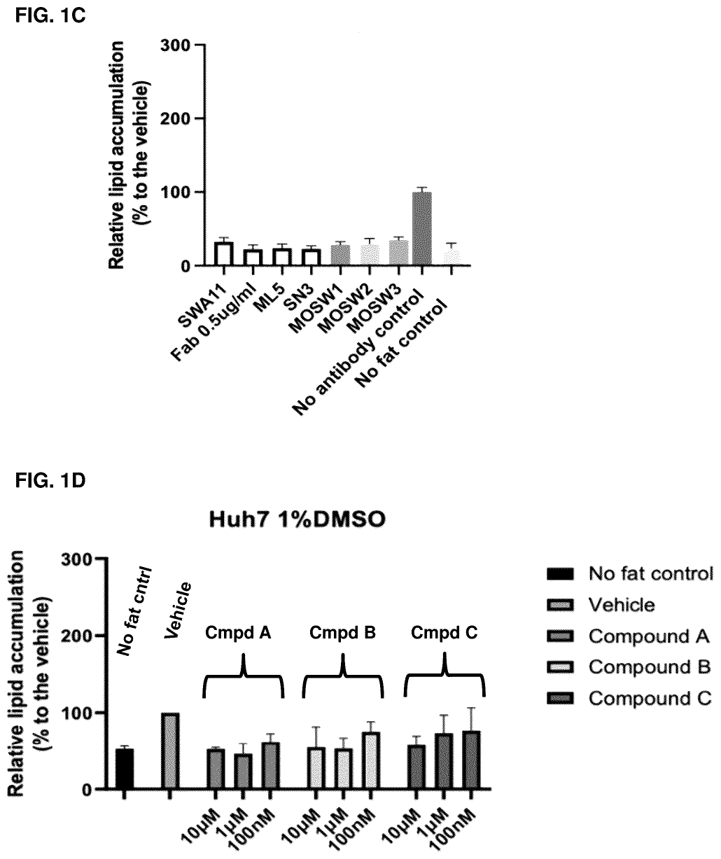

Anti-CD24 agents include, but are not limited to: antibodies against CD24 (with or without Fc effector function), fragment antigen-binding region (Fab) against CD24, single-chain variable fragment (scFv) against CD24, peptides that bind to CD24, RNA interference agents (siRNA/shRNA agents) against CD24, oligonucleotide antisense agents against CD24 (e.g., locked nucleic acid (LNA) targeting CD24 or any other oligonucleotide antisense), CRISPR agents targeting CD24 (e.g., CRISPR nuclease/cleavage activity to reduce CD24 expression at the DNA level via genome-editing, a CRISPRi agent to decrease CD24 expression, and the like), and small molecules that bind to CD24 (see, e.g., Compounds A, B, and C of FIG. 1). In some cases, an anti-CD24 agent is an antibody. In some cases it is a humanized antibody. Small molecules, peptides, DNA or RNA aptamers, and the like that target CD24 are also considered anti-CD24 agents.

Examples of CD24 specific binding members include CD24 antibodies and binding fragments thereof. Non-limiting examples of such antibodies include antibodies directed against any epitope of CD24. Also encompassed are bispecific antibodies, i.e., antibodies in which each of the two binding domains recognizes a different binding epitope.

CD24 is a two-chain glycosylphosphatidylinositol (GPI)—anchored glycoprotein expressed at multiple stages of B-cell development, beginning with the bone marrow pro-B-cell compartment and continuing through mature, surface Ig positive B-cells. Plasma cell expression is very low or negative. It is also expressed on the majority of B-lineage acute lymphoblastic leukemias, B-cell CCLs and B-cell non-Hodgkin's lymphomas. CD24 may play a role in regulation of B-cell proliferation and maturation. Protein references sequences include Genbank NP_001278666; NP_001278667; NP_001278668; NP_037362; NP_001346013.

Antibodies known to bind to human CD24 are known and commercially available, including, without limitation, SWA11 (Creative Biolab); MA5-11833; 12-0247-42; anti-CD24 clone ML5 (Biolegend), SN3 A5-2H10 (also referred to as SN3); ALB9, EPR19925; EPR3006 (N); SWA21; SWA22; OKB2, etc. An anti-CD24 agent may include, for example, an antibody that binds to human CD24, such as SN3 or ML5. G7s is a known scFv against human and mouse CD24. An anti-CD24 antibody may bind, for example, the LAP (leucine-alanine-proline) motif, as is known in the art. Alternatively, an antibody may be generated that is specific for human CD24.

Humanized anti-CD24 antibodies can be generated de novo as those disclosed here or are those known in the art include, for example, those described by Weber et al, Clinical Exp Immunol, 1993; Shapira et al. in: Proceedings of the 107th Annual Meeting of the American Association for Cancer Research; 2016 Apr. 16-20; New Orleans, LA. Philadelphia (PA): AACR; Cancer Res 2016; 76 (14 Suppl): Abstract nr 3805 and Sun et al. (2017) Oncotarget Vol. 8, (No. 31), pp: 51238-51252, each herein specifically incorporated by reference. Humanized anti-CD24 antibodies include, for example, humanized SWA11 antibody (as described in Arber patent U.S. Pat. No. 8,614,301 B2; incorporated by reference herein for its teachings related humanizing antibodies, e.g., the SWA11 antibody), and a humanized anti-CD24 antibody as described in international patent publication WO2019222082 (CN103819561A).

In some cases, an anti-CD24 agent includes an antigen binding region (of an antibody). In some cases, the antigen binding region includes the 3 heavy chain and 3 light chain CDRs (e.g., IMGT CDRs) of SWA11, ML5, SN3, or G7s (see, e.g., the IMGT CDRs of SWA11 of Table 1). In some cases, the antigen binding region includes the 3 heavy chain and 3 light chain CDRs (e.g., IMGT CDRs) of SWA11. In some cases, the antigen binding region includes the 3 heavy chain and 3 light chain CDRs (e.g., IMGT CDRs) of ML5. In some cases, the antigen binding region includes the 3 heavy chain and 3 light chain CDRs (e.g., IMGT CDRs) of SN3. In some cases, the antigen binding region includes the 3 heavy chain and 3 light chain CDRs (e.g., IMGT CDRs) of G7s.

In some cases, an anti-CD24 agent includes an antigen binding region that includes an amino acid sequence having the 3 heavy chain IMGT CDRs of SWA11 and having 80% or more (e.g., 85% or more, 90% or more, 95% or more, 97% or more, 98% or more, 99% or more, or 100%) sequence identity with the SWA11 variable heavy chain sequence of SEQ ID NO: 1; and an amino acid sequence having the 3 light chain IMGT CDRs of SWA11 and having 80% or more (e.g., 85% or more, 90% or more, 95% or more, 97% or more, 98% or more, 99% or more, or 100%) sequence identity with the SWA11 variable light chain sequence of SEQ ID NO: 18 (see, e.g., Table 1). In some cases, the antigen binding region includes an amino acid sequence having the 3 heavy chain IMGT CDRs of SWA11 and having 90% or more (e.g., 95% or more, 97% or more, 98% or more, 99% or more, or 100%) sequence identity with the SWA11 variable heavy chain sequence of SEQ ID NO: 1; and an amino acid sequence having the 3 light chain IMGT CDRs of SWA11 and having 90% or more (e.g., 95% or more, 97% or more, 98% or more, 99% or more, or 100%) sequence identity with the SWA11 variable light chain sequence of SEQ ID NO: 18. In some cases, the antigen binding region includes an amino acid sequence having the 3 heavy chain IMGT CDRs of SWA11 and having 98% or more (e.g., 99% or more, or 100%) sequence identity with the SWA11 variable heavy chain sequence of SEQ ID NO: 1; and an amino acid sequence having the 3 light chain IMGT CDRs of SWA11 and having 98% or more (e.g., 99% or more, or 100%) sequence identity with the SWA11 variable light chain sequence of SEQ ID NO: 18. In some cases, the antigen binding region includes the SWA11 variable heavy chain sequence of SEQ ID NO: 1; and the SWA11 variable light chain sequence of SEQ ID NO: 18.

In some cases, an anti-CD24 agent includes an antigen binding region that includes an amino acid sequence having the 3 heavy chain IMGT CDRs of SWA11 and having 80% or more (e.g., 85% or more, 90% or more, 95% or more, 97% or more, 98% or more, 99% or more, or 100%) sequence identity with the humanized variable heavy chain sequence of any one of SEQ ID NOs: 2-14; and an amino acid sequence having the 3 light chain IMGT CDRs of SWA11 and having 80% or more (e.g., 85% or more, 90% or more, 95% or more, 97% or more, 98% or more, 99% or more, or 100%) sequence identity with the humanized variable light chain sequence of any one of SEQ ID NOs: 19-31 (see, e.g., Table 1). In some cases, the antigen binding region includes an amino acid sequence having the 3 heavy chain IMGT CDRs of SWA11 and having 90% or more (e.g., 95% or more, 97% or more, 98% or more, 99% or more, or 100%) sequence identity with the humanized variable heavy chain sequence of any one of SEQ ID NOs: 2-14; and an amino acid sequence having the 3 light chain IMGT CDRs of SWA11 and having 90% or more (e.g., 95% or more, 97% or more, 98% or more, 99% or more, or 100%) sequence identity with the humanized variable light chain sequence of any one of SEQ ID NOs: 19-31. In some cases, the antigen binding region includes an amino acid sequence having the 3 heavy chain IMGT CDRs of SWA11 and having 98% or more (e.g., 99% or more, or 100%) sequence identity with the humanized variable heavy chain sequence of any one of SEQ ID NOs: 2-14; and an amino acid sequence having the 3 light chain IMGT CDRs of SWA11 and having 98% or more (e.g., 99% or more, or 100%) sequence identity with the humanized variable light chain sequence of any one of SEQ ID NOs: 19-31. In some cases, the antigen binding region includes the humanized variable heavy chain sequence of any one of SEQ ID NOs: 2-14; and the humanized variable light chain sequence of any one of SEQ ID NOs: 18-31.

The term “antibody” herein is used in the broadest sense and specifically covers monoclonal antibodies, polyclonal antibodies, monomers, dimers, multimers, multispecific antibodies (e.g., bispecific antibodies), heavy chain only antibodies, three chain antibodies, single chain Fv, single domain antibodies, nanobodies, etc., and also include antibody fragments with or without pegylation, so long as they exhibit the desired biological activity (Miller et al (2003) Jour. of Immunology 170:4854-4861). Antibodies may be murine, human, humanized, chimeric, or derived from other species. Antibodies, also referred to as immunoglobulins, conventionally comprise at least one heavy chain and one light, where the amino terminal domain of the heavy and light chains is variable in sequence, hence is commonly referred to as a variable region domain, or a variable heavy (VH) or variable light (VL) domain. The two domains conventionally associate to form a specific binding region. A “functional” or “biologically active” antibody or antigen-binding molecule is one capable of exerting one or more of its natural activities in structural, regulatory, biochemical or biophysical events. For example, a functional antibody or other binding molecule may have the ability to specifically bind an antigen and the binding may in turn elicit or alter a cellular or molecular event such as signaling transduction or phagocytosis. A functional antibody may also block ligand activation of a receptor or act as an agonist or antagonist or as an allosteric modulator.

The term antibody may reference a full-length heavy chain, a full length light chain, an intact immunoglobulin molecule; or an immunologically active portion of any of these polypeptides, i.e., a polypeptide that comprises an antigen binding site that immunospecifically binds an antigen of a target of interest or part thereof, such targets including but not limited to, infection cell or cells that produce autoimmune antibodies associated with an autoimmune disease. The immunoglobulin disclosed herein may comprise any suitable Fc region, including without limitation, human or other mammalian, e.g. cynomogulus, IgG, IgE, IgM, IgD, IgA, IgG1, IgG2, IgG3, IgG4, IgA1 and IgA2 or subclass of immunoglobulin molecule, including hybrid Igs, hybrid Fcs, and engineered subclasses with altered Fc portions that provide for reduced or enhanced effector cell activity. The immunoglobulins can be derived from any species.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al (1991) Sequences of Proteins of Immunological Interest, 5th Ed. Public Health Service, National Institutes of Health, Bethesda, Md.). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region may comprise amino acid residues from a “complementarity determining region” or “CDR”, and/or those residues from a “hypervariable loop”. “Framework Region” or “FR” residues are those variable domain residues other than the hypervariable region residues as herein defined.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations, which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method.

The antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al (1984) Proc. Natl. Acad. Sci. USA, 81:6851-6855). Chimeric antibodies of interest herein include “primatized” antibodies comprising variable domain antigen-binding sequences derived from a non-human primate (e.g., Old World Monkey, Ape etc) and human constant region sequences.

An “intact antibody chain” as used herein is one comprising a full length variable region and a full length constant region. An intact “conventional” antibody comprises an intact light chain and an intact heavy chain, as well as a light chain constant domain (CL) and heavy chain constant domains, CH1, hinge, CH2 and CH3 for secreted IgG. Other isotypes, such as IgM or IgA may have different CH and CL domains. The constant domains may be native sequence constant domains (e.g., human native sequence constant domains) or amino acid sequence variants thereof. The intact antibody may have one or more “effector functions” which refer to those biological activities attributable to the Fc constant region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); antibody-dependent cellular phagocytosis (ADCP) phagocytosis; and down regulation of cell surface receptors. Constant region variants include those that alter the effector profile, binding to Fc receptors, and the like.

Depending on the amino acid sequence of the constant domain of their heavy chains, intact antibodies can be assigned to different “classes.” There are five major classes of intact immunoglobulin antibodies: IgA, IgD, IgE, IgG, and IgM, and several of these may be further divided into “subclasses” (isotypes), e.g., IgG1, IgG2, IgG3, IgG4, IgA1, and IgA2. The heavy-chain constant domains that correspond to the different classes of antibodies are called a, 0, E, Y, and u, respectively. The subunit structures and three-dimensional configurations of different classes of immunoglobulins are well known. Ig forms include hinge-modifications or hingeless forms (Roux et al (1998) J. Immunol. 161:4083-4090; Lund et al (2000) Eur. J. Biochem. 267:7246-7256; US 2005/0048572; US 2004/0229310). The light chains of antibodies from any vertebrate species can be assigned to one of two clearly distinct types, called K and A, based on the amino acid sequences of their constant domains.

A “functional Fc region” possesses an “effector function” of a native-sequence Fc region. Exemplary effector functions include C1q binding; complement-dependent cytotoxicity (CDC); Fc-receptor binding; antibody-dependent cellular cytotoxicity (ADCC); antibody-dependent cellular phagocytosis (ADCP); down-regulation of cell-surface receptors (e.g., B-cell receptor), etc. Such effector functions generally require the Fc region to be interact with a receptor, e.g. the FcγRI; FcγRIIA; FcγRIIB1; FcγRIIB2; FcγRIIIA; FcγRIIIB receptors, and the recycling receptor, FcRn; and can be assessed using various assays as disclosed, for example, in definitions herein.

A “dead” or silenced Fc is one that has been mutagenized to retain activity with respect to, for example, prolonging serum half-life, but which does not bind to or activate the low and high affinity Fc receptors. As would be known to one of ordinary skill in the art, a variety of mutations can be used to silence the Fc for particular functions and any convenient mutations can be used when desired. Examples of silencing Fc mutations include, but are not necessarily limited to:
Human IgG1: L234A; L235A (LALA mutation)
Human IgG1: L234A; L235A; P329G
Human IgG4: S228P
Human IgG4: S228P, F234A, L235A
Human IgG4 S228P, L235E
Human IgG4 S228P, L235E, P329G
Mouse IgG2a: K320A, K322A
Mouse IgG2a: L234A, L235A, K320A, K322A, P329G

Antibody specific binding members that may be employed include full antibodies or immunoglobulins of any isotype, as well as fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. Also encompassed by the term are Fab′, Fv, F(ab′)2, and or other antibody fragments that retain specific binding to antigen, and monoclonal antibodies. An antibody may be monovalent or bivalent.

“Antibody fragments” comprise a portion of an intact antibody, for example, the antigen binding or variable region of the intact antibody. Examples of antibody fragments include Fab, Fab′, F(ab′)2, and Fv fragments; diabodies; linear antibodies (Zapata et al., Protein Eng. 8 (10): 1057-1062 (1995)); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)2 fragment that has two antigen combining sites and is still capable of cross-linking antigen.

“Fv” is the minimum antibody fragment which contains a complete antigen-recognition and -binding site. This region consists of a dimer of one heavy- and one light-chain variable domain in tight, non-covalent association. It is in this configuration that the three CDRS of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The “Fab” fragment also contains the constant domain of the light chain and the first constant domain (CH1) of the heavy chain. Fab fragments differ from Fab′ fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2.

“Single-chain Fv” or “sFv” antibody fragments comprise the VH and VL domains of antibody, wherein these domains are present in a single polypeptide chain. In some embodiments, the Fv polypeptide further comprises a polypeptide linker between the VH and VL domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

Antibodies that may be used in connection with the present disclosure thus can encompass monoclonal antibodies, polyclonal antibodies, bispecific antibodies, Fab antibody fragments, F(ab)2 antibody fragments, Fv antibody fragments (e.g., VH or VL), single chain Fv antibody fragments and dsFv antibody fragments. Furthermore, the antibody molecules may be fully human antibodies, humanized antibodies, or chimeric antibodies. In some embodiments, the antibody molecules are monoclonal, fully human antibodies.

The antibodies that may be used in connection with the present disclosure can include any antibody variable region, mature or unprocessed, linked to any immunoglobulin constant region. If a light chain variable region is linked to a constant region, it can be a kappa chain constant region. If a heavy chain variable region is linked to a constant region, it can be a human gamma 1, gamma 2, gamma 3 or gamma 4 constant region, more preferably, gamma 1, gamma 2 or gamma 4 and even more preferably gamma 1 or gamma 4.

In some embodiments, fully human monoclonal antibodies directed against CD24 are generated using transgenic mice carrying parts of the human immune system rather than the mouse system.

Minor variations in the amino acid sequences of antibodies or immunoglobulin molecules are encompassed by the present invention, providing that the variations in the amino acid sequence maintain at least 75%, e.g., at least 80%, 90%, 95%, or 99% of the sequence. In particular, conservative amino acid replacements are contemplated. Conservative replacements are those that take place within a family of amino acids that are related in their side chains. Whether an amino acid change results in a functional peptide can readily be determined by assaying the specific activity of the polypeptide derivative. Fragments (or analogs) of antibodies or immunoglobulin molecules, can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Sequence motifs and structural conformations may be used to define structural and functional domains in accordance with the invention.

In yet other embodiments, the Anti-CD24 agent is an agent that modulates, e.g., inhibits, CD24 activity by binding to CD24. For example, small molecules that bind to the CD24 and inhibit its activity are of interest. Naturally occurring or synthetic small molecule compounds of interest include numerous chemical classes, such as organic molecules, e.g., small organic compounds having a molecular weight of more than 50 and less than about 2,500 daltons. Candidate agents comprise functional groups for structural interaction with proteins, particularly hydrogen bonding, and typically include at least an amine, carbonyl, hydroxyl or carboxyl group, preferably at least two of the functional chemical groups. The candidate agents may include cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Such molecules may be identified, among other ways, by employing the screening protocols described below.

FIG. 1D demonstrates the anti-steatosis efficacy of 3 small molecules (Compounds A, B, and C) that were identified by screening for molecules that bind CD24 (see working examples below). This finding (in combination with other findings described herein, e.g., that antibodies with silenced Fc regions and antibodies without an Fc region also work as anti-steatosis agents) demonstrates that any agents that bind to CD24 or reduce CD24 expression (e.g., anti-CD24 CRISPR agents such as CRISPR nuclease or CRISPRi, anti-CD24 RNAi agents such as shRNA or siRNA, anti-CD24 oligonucleotide agents such as LNAs, and the like) can be used in the methods described herein (e.g., as an anti-steatosis agent).

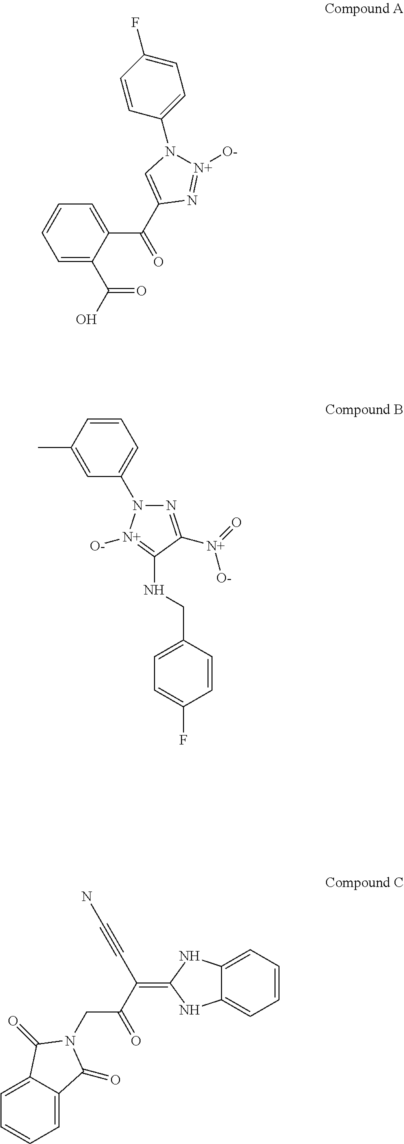 [SMILES: O=C(O)c1ccccc1C(=O)c1cn(-c2ccc(F)cc2)[n+]([O-])n1 ; Cc1cccc(-n2nc([N+](=O)[O-])c(NCc3ccc(F)cc3)[n+]2[O-])c1 ; NC#CC(C(=O)CN1C(=O)c2ccccc2C1=O)=C1Nc2ccccc2N1]

In those embodiments where an active agent is administered to the subject, the active agent(s) may be administered to the subject using any convenient administration protocol capable of resulting in the desired activity. Thus, the agent can be incorporated into a variety of formulations, e.g., pharmaceutically acceptable vehicles, for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments (e.g., skin creams), solutions, suppositories, injections, inhalants and aerosols. As such, administration of the agents can be achieved in various ways, including oral, buccal, rectal, parenteral, intraperitoneal, intradermal, transdermal, intracheal, etc., administration.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The agents can be utilized in aerosol formulation to be administered via inhalation. The compounds of the present invention can be formulated into pressurized acceptable propellants such as dichlorodifluoromethane, propane, nitrogen and the like.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Where the agent is a polypeptide, polynucleotide, analog or mimetic thereof, it may be introduced into tissues or host cells by any number of routes, including viral infection, microinjection, or fusion of vesicles. Jet injection may also be used for intramuscular administration, as described by Furth et al., Anal Biochem. (1992) 205:365-368. The DNA may be coated onto gold microparticles, and delivered intradermally by a particle bombardment device, or “gene gun” as described in the literature (see, for example, Tang et al., Nature (1992) 356:152-154), where gold microprojectiles are coated with the DNA, then bombarded into skin cells. For nucleic acid therapeutic agents, a number of different delivery vehicles find use, including viral and non-viral vector systems, as are known in the art.

Those of skill in the art will readily appreciate that dose levels can vary as a function of the specific compound, the nature of the delivery vehicle, and the like. Preferred dosages for a given compound are readily determinable by those of skill in the art by a variety of means.

An “effective amount” or “effective dose” refers to that amount which is capable of ameliorating or delaying progression of the diseased, degenerative or damaged condition. An effective amount can be determined on an individual basis and will be based, in part, on consideration of the symptoms to be treated and results sought. An effective amount can be determined by one of ordinary skill in the art employing such factors and using no more than routine experimentation.

In some cases, an effective amount will of an anti-CD24 agent will reduce steatosis (hepatic steatosis). In some cases, progression of steatosis will be blocked and in some cases steatosis will be reduced (i.e., the amount of steatosis observed will be less after administration of the anti-CD24 agent than was observed prior to administration). In some cases, steatosis will be reduced by 10% (that is, the amount observed will be 90% of what was present prior to administration) or more (e.g., 20% or more, 35% or more, 50% or more, 70% or more, 85% or more). In some cases, steatosis will be reduced by 30% (that is, the amount observed will be 70% of what was present prior to administration) or more (e.g., 50% or more, 70% or more, 85% or more). In some cases, steatosis will be reduced by 50% or more.

A “therapeutically effective dose” or “therapeutic dose” or “therapeutically effective amount” or “therapeutic amount” is an amount sufficient to effect desired clinical results (i.e., achieve therapeutic efficacy). A therapeutically effective dose can be administered in one or more administrations. In some embodiments the anti-CD24 agent is an antibody specific for human CD24, which optionally is a chimeric or humanized monoclonal antibody. In some embodiments an anti-CD24 antibody is administered at a dose of less than 8 mg/kg body weight, less than 2.5 mg/kg, less than 1 mg/kg, less than 0.75 mg/kg, less than 0.5 mg/kg, less than 0.25 mg/kg, less than 0.1 mg/kg, less than 0.05 mg/kg, less than 0.01 mg/kg. The therapeutic dose may be, for example, from 0.1 to 5 mg/kg, from 0.25 to 5 mg/kg, from 0.5 to 5 mg/kg, from 0.75 to 5 mg/kg, from 1 to 5 mg/kg; or from 0.1 to 2.5 mg/kg, from 0.25 to 2.5 mg/kg, from 0.5 to 2.5 mg/kg, from 0.75 to 2.5 mg/kg; from 0.1 to 1 mg/kg, from 0.25 to 1 mg/kg, from 0.5 to 1 mg/kg, from 0.75 to 1 mg/kg, etc.

In some embodiments the anti-CD24 agent is an antibody specific for human CD24, which optionally is a humanized or fully human monoclonal antibody. In some embodiments an anti-CD24 antibody is administered at a dose of less than 10 mg/kg body weight, less than 8 mg/kg, less than 2.5 mg/kg, less than 1 mg/kg, less than 0.75 mg/kg, less than 0.5 mg/kg, less than 0.25 mg/kg, less than 0.1 mg/kg, less than 0.5 mg/kg, less than 0.1 mg/kg. The therapeutic dose may be, for example, from 0.1 to 5 mg/kg, from 0.25 to 5 mg/kg, from 0.5 to 5 mg/kg, from 0.75 to 5 mg/kg, from 1 to 5 mg/kg; or from 0.1 to 2.5 mg/kg, from 0.25 to 2.5 mg/kg, from 0.5 to 2.5 mg/kg, from 0.7 to 2.5 mg/kg; from 0.1 to 1 mg/kg, from 0.25 to 1 mg/kg, from 0.5 to 1 mg/kg, from 0.75 to 1 mg/kg, etc.

In some embodiments the anti-CD24 agent is an antibody specific for human CD24 whose Fc has been silent (i.e., whose Fc is dead). Silencing mutations will be known to one of ordinary skill in the art and any convenient silencing mutation can be used. Examples of silencing Fc mutations include, but are not necessarily limited to: L234A/L235A (LALA mutation) (human IgG1), L234A/L235A/P329G (human IgG1), S228P (human IgG4), S228P/F234A/L235A (human IgG4), S228P/L235E (human IgG4), S228P/L235E/P329G (human IgG4), K320A/K322A (Mouse IgG2a), and L234A/L235A/K320A/K322A/P329G (Mouse IgG2a).

In some embodiments, an Fc-silent anti-CD24 antibody can be administered at a higher dose than the non Fc-silent anti-CD24 antibody, such that it can be administered at a dose of less than 100 mg/kg body weight, less than 90 mg/kg body weight, less than 80 mg/kg body weight, less than 70 mg/kg body weight, less than 60 mg/kg body weight, less than 50 mg/kg, less than 40 mg/kg body weight, less than 30 mg/kg body weight, less than 20 mg/kg body weight, less than 10 mg/kg body weight, less than 8 mg/kg, less than 2.5 mg/kg, less than 1 mg/kg, less than 0.75 mg/kg, less than 0.5 mg/kg, less than 0.25 mg/kg, less than 0.1 mg/kg, less than 0.5 mg/kg, less than 0.1 mg/kg. The therapeutic dose may be, for example, from 0.1 to 5 mg/kg, from 0.25 to 5 mg/kg, from 0.5 to 5 mg/kg, from 0.75 to 5 mg/kg, from 1 to 5 mg/kg; or from 0.1 to 2.5 mg/kg, from 0.25 to 2.5 mg/kg, from 0.5 to 2.5 mg/kg, from 0.7 to 2.5 mg/kg; from 0.1 to 1 mg/kg, from 0.25 to 1 mg/kg, from 0.5 to 1 mg/kg, from 0.75 to 1 mg/kg, etc. In some embodiments the anti-CD24 agent is a small molecule (see, e.g., compounds A, B, and C). An effective dose can be expected to be in a range of from about 0.001 to about 100 mg/kg body weight per day (for humans), in some cases from about 0.1 to about 50 mg/kg body weight per day, in some cases from about 1 to about 50 mg/kg body weight per day, in some cases about 5 to about 40 mg/kg body weight per day, in some cases about 2 to about 15 mg/kg body weight per day, and in some cases about 25 to about 40 mg/kg bodyweight per day.

Dosage and frequency may vary depending on the half-life of the anti-CD24 agent. It will be understood by one of skill in the art that such guidelines will be adjusted for the molecular weight of the active agent, e.g. in the use of antibody fragments, in the use of antibody conjugates, in the use of anti-CD24 agents etc. The dosage may also be varied for localized administration, e.g. intranasal, inhalation, etc., or for systemic administration, e.g. i.m., i.p., i.v., s.c., and the like.

The treatment course may be less than about 12 weeks, less than about 8 weeks, less than about 4 weeks, and may be, for example, from 1-12 weeks, from 2-12 weeks, from 4-12 weeks, from 4-8 weeks, etc. Administration may be once a week, twice a week, every other day, daily, twice a day, every two weeks, etc., and in some embodiments is once a week. In some embodiments, more than one course of treatment is administered. In some cases, the course of treatment is in a range of from 1-24 weeks (e.g., from 1-8 weeks, 1-4 weeks, about 1 week, about 2 weeks, or about 3 weeks). In some cases, the course of treatment is in a range of from 1 day-8 weeks (e.g., from 1 day-6 weeks, 1 day-4 weeks, 1 day-2 weeks, 1 day-7 days, 3 days-8 weeks, 3 days-6 weeks, 3 days-4 weeks, 3 days-2 weeks, or 3 days-7 days). In some cases, the compound is administered for 2 or more days (e.g., 3 or more, 4 or more, 5 or more, 6 or more, or 7 or more days).

An anti-CD24 agent can be administered by any suitable means, including topical, oral, parenteral, intrapulmonary, and intranasal. Parenteral infusions include intramuscular, intravenous (bolus or slow drip), intraarterial, intraperitoneal, intrathecal or subcutaneous administration. An anti-CD24 agent can be administered in any manner which is medically acceptable. This may include injections, by parenteral routes such as intravenous, intravascular, intraarterial, subcutaneous, intramuscular, intratumor, intraperitoneal, intraventricular, intraepidural, or others as well as oral, nasal, ophthalmic, rectal, or topical. Sustained release administration is also specifically included in the disclosure, by such means as depot injections or erodible implants. Localized delivery is particularly contemplated, by such means as delivery via a catheter to one or more arteries, such as the renal artery or a vessel supplying a localized tumor.

Administration of an anti-CD24 agent may be combined with other therapeutic modalities to decrease fatty liver. Such modalities can include but are not limited to modulating alcohol consumption, dietary changes, exercise, other agents to treat fatty liver such as metformin, vitamin E, and the like. The terms “co-administration”, “co-administer”, and “in combination with” include the administration of two or more therapeutic agents (e.g., an anti-CD24 agent and an additional agent, two different anti-CD24 agents, and the like) either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent. Administration of an anti-CD24 agent may be combined with co-administration of any number of additional agents. An anti-CD24 agent need not be, but is optionally formulated with one or more agents that potentiate activity, or that otherwise increase the therapeutic effect. These are generally used in the same dosages and with administration routes as used herein or from 1 to 99% of the heretofore employed dosages. In some embodiments, treatment is accomplished by administering a combination (co-administration) of a subject anti-CD24 agent and/or another agent that opsonizes a target cell.

Treatment may also be combined with other active agents, such as antibiotics, cytokines, etc. Classes of antibiotics include penicillins, e.g. penicillin G, penicillin V, methicillin, oxacillin, carbenicillin, nafcillin, ampicillin, etc.; penicillins in combination with β-lactamase inhibitors, cephalosporins, e.g. cefaclor, cefazolin, cefuroxime, moxalactam, etc.; carbapenems; monobactams; aminoglycosides; tetracyclines; macrolides; lincomycins; polymyxins; sulfonamides; quinolones; cloramphenical; metronidazole; spectinomycin; trimethoprim; vancomycin; etc. Cytokines may also be included, e.g. interferon γ, tumor necrosis factor α, interleukin 12, etc.

Examples of diseases/ailments that can be treated with an anti-CD24 agent include, but are not limited to: (1) obesity; (2) obesity-related diseases such as hypertension, dyslipidemia, coronary heart disease, gallbladder disease, osteoarthritis, sleep apnea and breathing problems, mental illness such as clinical depression, anxiety, and other mental disorders, ischemic cardiovascular disorders, hyperlipidemia, hyperuricemia, insulin resistance, noninsulin dependent diabetes mellitus (NIDDM, or Type II diabetes), insulin dependent diabetes mellitus (IDDM or Type I diabetes), diabetes-related complications including microangiopathic lesions, ocular lesions, retinopathy, neuropathy, and renal lesions, cardiovascular disease (including cardiac insufficiency, coronary insufficiency, and high blood pressure), atherosclerosis, atheromatous disease, stroke, Syndrome X, forms of cancer such as uterine, breast, colorectal, kidney, and gallbladder, and high cholesterol levels, (3) fatty liver (e.g., Nonalcoholic Fatty Liver Disease (NAFLD) and Nonalcoholic steatohepatitis (NASH)); and (4) fatty liver associated diseases such as obesity with high level of belly fat, high blood pressure, diabetes (type 2), high choloesteral, high triglyceride levels, and sleep apnea.

In some embodiments, the individual is one who is receiving a liver transplant and the anti-CD24 agent is used to enhance liver graft function and/or prolong graft survival.

### Kits

Also provided are kits for use in the methods. The subject kits can include an anti-CD24 agent. In some embodiments, an anti-CD24 agent is provided in a dosage form (e.g., a therapeutically effective dosage form. In the context of a kit, an anti-CD24 agent can be provided in liquid or solid form in any convenient packaging (e.g., stick pack, dose pack, etc.). The agents of a kit can be present in the same or separate containers. The agents may also be present in the same container. In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address.

### Exemplary Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below (see SET A and SET B). As will be apparent to those of ordinary skill in the art upon reading this disclosure, each of the individually numbered aspects may be used or combined with any of the preceding or following individually numbered aspects. This is intended to provide support for all such combinations of aspects and is not limited to combinations of aspects explicitly provided below. It will be apparent to one of ordinary skill in the art that various changes and modifications can be made without departing from the spirit or scope of the invention.

### SET A

1. A method of treating obesity using anti-CD24 agents.
2. A method of treating obesity-related diseases using anti-CD24 agents.
3. A method of treating fatty liver and fatty liver associated diseases using anti-CD24 agents.
4. A method of 2, where the obesity-related diseases is non-alcoholic fatty liver disease (NAFLD)
5. A method of 3, where the fatty liver and fatty liver associated disease is non-alcoholic fatty liver disease (NAFLD)
6. A method of reducing steatosis in an individual, the method comprising: contacting the individual with a therapeutically effective dose of an anti-CD24 agent for a period of time sufficient to reduce steatosis.
7. A method of administering anti-CD24 agent to a patient receiving a liver transplant to enhance liver graft function
8. A method of administering anti-CD24 agent to a patient receiving a liver transplant to prolong graft survival.
9. A method of any 1-8, where anti-CD24 agent is elected from: antibodies against CD24, antibodies against CD24 whose Fc is engineered to have silent ADCC and/or ADCP and/or complement binding activity, fragment antigen-binding region (Fab) against CD24 or pegylated Fab fragment against CD24, single-chain variable fragment (scFv) against CD24, peptides that bind CD24, small molecules that bind to CD24.
10. The method of any of 1-9, wherein the course of treatment is from 1-24 weeks.
11. The method of any of 1-9, wherein the course of treatment is from 1-8 weeks.
12. The method of any of 1-9, wherein the course of treatment is from 1-4 weeks.
13. The method of any of 1-9, wherein the course of treatment is for 1 week.
14. The method of any of 1-9, wherein anti-CD24 agent can be combined with another therapeutic modality including but not limited to: dietary modification, weight loss surgery, or other therapeutics.
15. The method of any of 1-14 where the anti-CD24 agent is an anti-CD24 antibody that binds to leucine-alanine-proline motif of CD24.
16. The method of any of 1-14 where the anti-CD24 agent is a humanized anti-CD24 antibody.

### SET B

1. A method of reducing steatosis in an individual, the method comprising: administering to an individual who has steatosis, a therapeutically effective dose of an anti-CD24 agent for a period of time sufficient to reduce the steatosis.
2. The method of 1, wherein the subject has non-alcoholic fatty liver disease (NAFLD) or non-alcoholic steatohepatitis (NASH).
3. The method of 1 or 2, wherein the anti-CD24 agent is an anti-CD24 antibody.
4. The method of 3, wherein said anti-CD24 antibody comprises an Fc with one or more silencing mutations.
5. The method of 3 or 4, wherein said anti-CD24 antibody is an anti-CD24 fragment antigen-binding region (Fab) or an anti-CD24 pegylated Fab fragment.
6. The method of 3 or 4, wherein said anti-CD24 antibody is an anti-CD24 single-chain variable fragment (scFv).
7. The method of any one of 3-6, wherein the anti-CD24 antibody comprises the 3 heavy chain and 3 light chain CDRs of SWA11, ML5, SN3, or G7s.
8. The method of any one of 3-7, wherein the anti-CD24 antibody is a humanized antibody.
9. The method of any one of 3-6, wherein the anti-CD24 antibody comprises an amino acid sequence having the 3 heavy chain IMGT CDRs of SWA11 and having 80% or more sequence identity with the humanized variable heavy chain sequence of any one of SEQ ID NOs: 2-14; and an amino acid sequence having the 3 light chain IMGT CDRs of SWA11 and having 80% or more sequence identity with the humanized variable light chain sequence of any one of SEQ ID NOs: 19-31.
10. The method of 1 or 2, wherein the anti-CD24 agent is a small molecule that binds to CD24.
11. The method of any of 1-10, wherein the course of treatment is from 1-8 weeks.
12. The method of any of 1-11, wherein the said administering is combined with dietary modification and/or weight loss surgery.
13. The method of any of 1-12, wherein the anti-CD24 agent is co-administered with another therapeutic agent.
14. The method of any of 1-13, wherein the individual is a liver transplant patient and said administering is to enhance liver graft function and/or prolong liver graft survival.
15. The method of any of 1-14, wherein the individual has fatty liver, a fatty liver associated disease, obesity, or an obesity-related disease.

The following example(s) is/are offered by way of illustration and not by way of limitation.

### Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like.

Cultured cells when incubated with fatty acids can have increased fatty infiltrates, also known as steatosis. Here we showed that this cellular fatty infiltrate can be reversed upon treating with an anti-CD24 agent. anti-CD24 agents may include but are not limited to antibodies against CD24, fragment antigen-binding region (Fab) against CD24, single-chain variable fragment (scFv) against CD24, peptides that bind to CD24, siRNA/shRNA against CD24, oligonucleotide antisense against CD24 such as locked nucleic acid (LNA) targeting CD24 or any other oligonucleotide antisense, CRISPR against CD24, and small molecules that bind to CD24. We then sought to determine the therapeutic efficacy of anti-CD24 agent in various in vivo models of fatty liver. Mice can develop fatty liver when placed on a high fat diet. Mice with certain genetic mutations such as those who are homozygous for the obese spontaneous mutation (ob), also known as ob/ob mice (The Jackson Laboratory) develop steatosis on a regular diet. In addition, these ob/ob mice gain weight rapidly and exhibit features of metabolic syndrome such as hyperglycemia and glucose intolerance. In ob/ob mice, hepatic steatosis can further be accelerated and made more severe when they are placed on a high fat diet. Using either in vivo model, we showed that anti-CD24 agents have pronounced therapeutic efficacy in reducing hepatic steatosis. In addition, such treatment can lead to decreased liver enzymes such as ALT, AST, alkaline phosphatase, and GGT and leads to improved liver function. To further demonstrate the therapeutic efficacy of anti-CD24 agent in an in vivo model that can more physiologically recapitulate human fatty liver disease, we utilized an in vivo model where NSG mice can be engrafted with human liver cells. When these mice are placed on a high fat diet, we showed that treatment with anti-CD24 was also able to combat steatosis and improve graft function and graft survival.

### Administration of Anti-CD24 Agents Inhibits Hepatic Steatosis Development in Cell Culture

We identified the anti-steatosis efficacy of therapeutics binding to CD24 in a cell culture system. Specifically, Huh7 cells, which are liver hepatoma cells, were cultured in 1% DMSO (Huh7D cells) and then supplemented with fatty medium to induce steatosis. Fatty medium was prepared by 1:1 mixture of oleic acid (Sigma Aldrich) and palmitic acid (Sigma Aldrich) with 10% FFA-free bovine serum albumin (BSA) solution (Sigma Aldrich) in PBS following previous literature (35). Briefly, 100 mM of oleic acid and palmitic acid was prepared in 99% methanol. Palmitic acid was prepared by shaking at 500 rpm 50° C. for 20 mins. 10 mM of oleic acid and palmitic acid was conjugated with 10% BSA by incubating at 50° C. for 30 mins. 100 μM FFA mixture was dissolved in hepatocyte medium. Cells were incubated in fatty medium for 4 days to induce fatty liver. Cells were then treated with anti-CD24 agents for 3 days in the presence of the fatty medium. To determine the degree of steatosis, cells were stained with 1 mg/ml Nile Red solution (Sigma Aldrich) in cell culture medium for 15 mins at 37° C. and florescence signal was measured. Treatment was then done with a wide variety of anti-CD24 agents including antibodies against CD24, the Fragment-antigen-binding (Fab) against CD24, humanized antibodies against CD24, and small molecules that bind CD24, all of which exhibited anti-steatosis efficacy (see FIG. 1).

TABLE 1
Sequences of antibodies used in FIG. 1 (IMGT CDRs are bold and underlined)

| | | SEQ ID | | SEQ ID |
|---|---|---|---|---|
| Name | VH Seq | NO: | VL Seq | NO: |
| SWA11 | DVQLQESGPDLVKPSQSLSLTCTV | 1 | DIVMSQSPSSLNVSVGEKVTMRCRSSQ | 18 |
| | TGYSITSGYTWHWIRQFPGNTVE | | SLLYSSDQKNYLTWYQQKPGQSPKLLIS | |
| | WMGYIQYTGSTRYNPALRGRLSIS | | WASTRASGVPDRFTGSGSGTDFTLTISS | |
| | RDTSKNQFFLQLISVTTADTGTYFC | | VKAEDLGVYYCQQYFIYPLTFGVGTKLG | |
| | ARGTTASFDYWGQGTTLTVASA | | LK | |
| V2 | DVQLQESGPGLVKPSQTLSLTCTV | 2 | DIVMTQSPLSLPVTPGEPASISCRSSQSL | 19 |
| | TGYSITSGYTWHWIRQPPGKGLE | | LYSSDQKNYLTWYLQKPGQSPQLLISW | |
| | WMGYIQYTGSTRYNPSLKSRVTIS | | ASTRASGVPDRFSGSGSGTDFTLKISRV | |
| | RDTSKNQFSLKLSSVTAADTAVYF | | EAEDVGVYYCQQYFIYPLTFGGGTKLEI | |
| | CARGTTASFDYWGQGTTVTVSS | | K | |
| V3 | DVQLQESGPGLVKPSQTLSLTCTV | 3 | DIQMTQSPSSLSASVGDRVTITCRSSQS | 20 |
| | TGYSITSGYTWHWIRQPPGKGLE | | LLYSSDQKNYLTWYQQKPGKAPKLLISW | |
| | WMGYIQYTGSTRYNPSLKSRVTIS | | ASTRASGVPSRFSGSGSGTDFTLTISSL | |
| | RDTSKNQFSLKLSSVTAADTAVYF | | QPEDFATYYCQQYFIYPLTFGGGTKLEIK | |
| | CARGTTASFDYWGQGTTVTVSS | | | |
| V4 | DVQLQESGPGLVKPSQTLSLTCTV | 4 | DIQMTQSPSSLSASVGDRVTITCRSSQS | 21 |
| | TGYSITSGYTWHWIRQPPGKGLE | | LLYSSDQKNYLTWYQQKPGKAPKLLISW | |
| | WMGYIQYTGSTRYNPSLKSRVTIS | | ASTLASGVPSRFSGSGSGTDFTLTISSL | |
| | RDTSKNQFSLKLSSVTAADTAVYF | | QPEDFATYYCQQYFIYPLTFGGGTKLEIK | |
| | CARGTTASFDYWGQGTTVTVSS | | | |
| V6 | QVQLQESGPGLVKPSQTLSLTCTV | 5 | DIVMTQSPLSLPVTPGEPASISCRSSQSL | 22 |
| | TGYSITSGYTWSWIRQPPGKGLE | | LYSSDQKNYLTWYLQKPGQSPQLLISW | |
| | WIGYIQYTGSTRYNPSLKSRVTISR | | ASTRASGVPDRFSGSGSGTDFTLKISRV | |
| | DTSKNQFSLKLSSVTAADTAVYYC | | EAEDVGVYYCQQYFIYPLTFGGGTKLEI | |
| | ARGTTASFDYWGQGTTVTVSS | | K | |
| V7 | QVQLQESGPGLVKPSQTLSLTCTV | 6 | DIQMTQSPSSLSASVGDRVTITCRSSQS | 23 |
| | TGYSITSGYTWSWIRQPPGKGLE | | LLYSSDQKNYLTWYQQKPGKAPKLLISW | |
| | WIGYIQYTGSTRYNPSLKSRVTISR | | ASTRASGVPSRFSGSGSGTDFTLTISSL | |
| | DTSKNQFSLKLSSVTAADTAVYYC | | QPEDFATYYCQQYFIYPLTFGGGTKLEIK | |
| | ARGTTASFDYWGQGTTVTVSS | | | |
| V8 | QVQLQESGPGLVKPSQTLSLTCTV | 7 | DIQMTQSPSSLSASVGDRVTITCRSSQS | 24 |
| | TGYSITSGYTWSWIRQPPGKGLE | | LLYSSDQKNYLTWYQQKPGKAPKLLISW | |
| | WIGYIQYTGSTRYNPSLKSRVTISR | | ASTLASGVPSRFSGSGSGTDFTLTISSL | |
| | DTSKNQFSLKLSSVTAADTAVYYC | | QPEDFATYYCQQYFIYPLTFGGGTKLEIK | |
| | ARGTTASFDYWGQGTTVTVSS | | | |
| V9 | DVQLKESGPALVKPTQTLTLTCTV | 8 | DIVMTQSPDSLAVSLGERATINCKSSQS | 25 |
| | TGYSITSGYTWHWIRQPPGKALE | | LLYSSDQKNYLTWYQQKPGQPPKLLIS | |
| | WMGYIQYTGSTRYNPSLKTRLTIS | | WASTRASGVPDRFSGSGSGTDFTLTISS | |
| | RDTSKNQFVLTMTNMDPVDTATYF | | LQAEDVAVYYCQQYFIYPLTFGGGTKLEI | |
| | CARGTTASFDYWGQGTTVTVSS | | K | |
| V10 | DVQLKESGPALVKPTQTLTLTCTV | 9 | DIVMTQSPLSLPVTPGEPASISCRSSQSL | 26 |
| | TGYSITSGYTWHWIRQPPGKALE | | LYSSDQKNYLTWYLQKPGQSPQLLISW | |
| | WMGYIQYTGSTRYNPSLKTRLTIS | | ASTRASGVPDRFSGSGSGTDFTLKISRV | |
| | RDTSKNQFVLTMTNMDPVDTATYF | | EAEDVGVYYCQQYFIYPLTFGGGTKLEI | |
| | CARGTTASFDYWGQGTTVTVSS | | K | |
| V14 | QVTLKESGPALVKPTQTLTLTCTVT | 10 | DIVMTQSPLSLPVTPGEPASISCRSSQSL | 27 |
| | GYSITSGYTVSWIRQPPGKALEWI | | LYSSDQKNYLTWYLQKPGQSPQLLISW | |
| | GYIQYTGSTRYNPSLKTRLTISRDT | | ASTRASGVPDRFSGSGSGTDFTLKISRV | |
| | SKNQFVLTMTNMDPVDTATYYCA | | EAEDVGVYYCQQYFIYPLTFGGGTKLEI | |
| | RGTTASFDYWGQGTTVTVSS | | K | |
| V15 | QVTLKESGPALVKPTQTLTLTCTVT | 11 | DIQMTQSPSSLSASVGDRVTITCRSSQS | 28 |
| | GYSITSGYTVSWIRQPPGKALEWI | | LLYSSDQKNYLTWYQQKPGKAPKLLISW | |
| | GYIQYTGSTRYNPSLKTRLTISRDT | | ASTRASGVPSRFSGSGSGTDFTLTISSL | |
| | SKNQFVLTMTNMDPVDTATYYCA | | QPEDFATYYCQQYFIYPLTFGGGTKLEIK | |
| | RGTTASFDYWGQGTTVTVSS | | | |
| V16 | QVTLKESGPALVKPTQTLTLTCTVT | 12 | DIQMTQSPSSLSASVGDRVTITCRSSQS | 29 |
| | GYSITSGYTVSWIRQPPGKALEWI | | LLYSSDQKNYLTWYQQKPGKAPKLLISW | |
| | GYIQYTGSTRYNPSLKTRLTISRDT | | ASTLASGVPSRFSGSGSGTDFTLTISSL | |
| | SKNQFVLTMTNMDPVDTATYYCA | | QPEDFATYYCQQYFIYPLTFGGGTKLEIK | |
| | RGTTASFDYWGQGTTVTVSS | | | |
| TW2 | QVQLQESGPGLVKPSQTLSLTCTV | 13 | DIVMTQSPDSLAVSLGERATINCKSSQS | 30 |
| | TGYSITSGYTWSWIRQHPGKGLE | | LLYSSDQKNYLAWYQQKPGQSPKLLIY | |
| | WMGYIQYTGSTRYNPSLKSLLTIS | | WASTRESGVPDRFSGSGSGTDFTLTISS | |
| | RDTSKNQFSLKLSSVTAADTAVYY | | LQAEDVAVYYCQQYFIYPLTFGQGTKLEI | |
| | CARGTTASFDYWGQGTTVTVSS | | K | |
| TW6 | QVQLQESGPGLVKPSQTLSLTCTV | 14 | DIVMTQSPDSLAVSLGERATINCKSSQS | 31 |
| | TGYSITSGYTWSWIRQHPGKGLE | | LLYSSDQKNYLAWYQQKPGQSPKLLIY | |
| | WMGYIQYTGSTRYNPSLKSLVTIS | | WASTRESGVPDRFSGSGSGTDFTLTISS | |
| | RDTSKNQFSLKLSSVTAADTAVYY | | LQAEDVAVYYCQQYFIYPLTFGQGTKLEI | |
| | CARGTTASFDYWGQGTTVTVSS | | K | |

| | | |
|---|---|---|
| Mouse IgG2a- | ADCP+++ | Heavy Chain: |
| kappa-SWA-11 | ADCC+++* | DVQLQESGPDLVKPSQSLSLTCTVTGYSITSGYTWHWIRQFPGNTV |
| ″MOSW1″ | CDC+++ | EWMGYIQYTGSTRYNPALRGRLSISRDTSKNQFFLQLISVTTADTGT |
| | | YFCARGTTASFDYWGQGTTLTVASAKTTAPSVYPLAPVCGDTTGSS |
| | | VTLGCLVKGYFPEPVTLTWNSGSLSSGVHTFPAVLQSDLYTLSSSVT |
| | | VTSSTWPSQSITCNVAHPASSTKVDKKIEPRGPTIKPCPPCKCPAPN |
| | | LLGGPSVFIFPPKIKDVLMISLSPIVTCVVVDVSEDDPDVQISWFVNNV |
| | | EVHTAQTQTHREDYNSTLRVVSALPIQHQDWMSGKEFKCKVNNKD |
| | | LPAPIERTISKPKGSVRAPQVYVLPPPEEEMTKKQVTLTCMVTDFMP |
| | | EDIYVEWTNNGKTELNYKNTEPVLDSDGSYFMYSKLRVEKKNWVER |
| | | NSYSCSVVHEGLHNHHTTKSFSRTPGK |
| | | (SEQ ID NO: 15) |
| | | Light Chain: |
| | | DIVMSQSPSSLNVSVGEKVTMRCRSSQSLLYSSDQKNYLTWYQQK |
| | | PGQSPKLLISWASTRASGVPDRFTGSGSGTDFTLTISSVKAEDLGVY |
| | | YCQQYFIYPLTFGVGTKLGLKRADAAPTVSIFPPSSEQLTSGGASVV |
| | | CFLNNFYPKDINVKWKIDGSERQNGVLNSWTDQDSKDSTYSMSSTL |
| | | TLTKDEYERHNSYTCEATHKTSTSPIVKSFNRNEC |
| | | (SEQ ID NO: 32) |
| Mouse IgG2a | ADCP+++ | Heavy Chain: |
| lacking mouse | ADCC+++* | DVQLQESGPDLVKPSQSLSLTCTVTGYSITSGYTWHWIRQFPGNTV |
| C1q binding | CDC− | EWMGYIQYTGSTRYNPALRGRLSISRDTSKNQFFLQLISVTTADTGT |
| (retains Fc | | YFCARGTTASFDYWGQGTTLTVASAKTTAPSVYPLAPVCGDTTGSS |
| activity) | | VTLGCLVKGYFPEPVTLTWNSGSLSSGVHTFPAVLQSDLYTLSSSVT |
| (K320A, K322A) | | VTSSTWPSQSITCNVAHPASSTKVDKKIEPRGPTIKPCPPCKCPAPN |
| “MOSW2” | | LLGGPSVFIFPPKIKDVLMISLSPIVTCVVVDVSEDDPDVQISWFVNNV |
| | | EVHTAQTQTHREDYNSTLRVVSALPIQHQDWMSGKEFACAVNNKD |
| | | LPAPIERTISKPKGSVRAPQVYVLPPPEEEMTKKQVTLTCMVTDFMP |
| | | EDIYVEWTNNGKTELNYKNTEPVLDSDGSYFMYSKLRVEKKNWVER |
| | | NSYSCSVVHEGLHNHHTTKSFSRTPGK |
| | | (SEQ ID NO: 16) |
| | | Light Chain: |
| | | DIVMSQSPSSLNVSVGEKVTMRCRSSQSLLYSSDQKNYLTWYQQK |
| | | PGQSPKLLISWASTRASGVPDRFTGSGSGTDFTLTISSVKAEDLGVY |
| | | YCQQYFIYPLTFGVGTKLGLKRADAAPTVSIFPPSSEQLTSGGASVV |
| | | CFLNNFYPKDINVKWKIDGSERQNGVLNSWTDQDSKDSTYSMSSTL |
| | | TLTKDEYERHNSYTCEATHKTSTSPIVKSFNRNEC |
| | | (SEQ ID NO: 33) |
| Mouse IgG2a- | ADCP− | Heavy Chain: |
| C1q and Fc | ADCC− | DVQLQESGPDLVKPSQSLSLTCTVTGYSITSGYTWHWIRQFPGNTV |
| silenced | CDC− | EWMGYIQYTGSTRYNPALRGRLSISRDTSKNQFFLQLISVTTADTGT |
| (L234A, L235A, | | YFCARGTTASFDYWGQGTTLTVASAKTTAPSVYPLAPVCGDTTGSS |
| K320A, K322A | | VTLGCLVKGYFPEPVTLTWNSGSLSSGVHTFPAVLQSDLYTLSSSVT |
| P329G) | | VTSSTWPSQSITCNVAHPASSTKVDKKIEPRGPTIKPCPPCKCPAPN |
| “MOSW3” | | AAGGPSVFIFPPKIKDVLMISLSPIVTCVVVDVSEDDPDVQISWFVNN |
| | | VEVHTAQTQTHREDYNSTLRVVSALPIQHQDWMSGKEFACAVNNK |
| | | DLGAPIERTISKPKGSVRAPQVYVLPPPEEEMTKKQVTLTCMVTDFM |
| | | PEDIYVEWTNNGKTELNYKNTEPVLDSDGSYFMYSKLRVEKKNWVE |
| | | RNSYSCSVVHEGLHNHHTTKSFSRTPGK |
| | | (SEQ ID NO: 17) |
| | | Light Chain: |
| | | DIVMSQSPSSLNVSVGEKVTMRCRSSQSLLYSSDQKNYLTWYQQK |
| | | PGQSPKLLISWASTRASGVPDRFTGSGSGTDFTLTISSVKAEDLGVY |
| | | YCQQYFIYPLTFGVGTKLGLKRADAAPTVSIFPPSSEQLTSGGASVV |
| | | CFLNNFYPKDINVKWKIDGSERQNGVLNSWTDQDSKDSTYSMSSTL |
| | | TLTKDEYERHNSYTCEATHKTSTSPIVKSFNRNEC |
| | | (SEQ ID NO: 34) |

With regard to the small molecules (Compounds A, B, and C of FIG. 1D): Virtual screening of diverse chemical libraries was performed against human CD24. Briefly, the sequence corresponding to the mature extracellular domain of human CD24 was folded using alphafold2. Docking was performed using the Schrödinger software suite (Maestro, version 12.4, Schrödinger New York, NY, USA) following well-established approaches. Diverse compound libraries were extracted into SDF format and imported into Maestro and prepared for docking using LigPrep. loniser was used to generate an ionized state of all compounds at the target pH 7±2. The folded human CD24 sequence was prepared with Protein Preparation Wizard where hydrogens and bond orders were assigned, heteroatomic states were generated and restrained minimization with OPLS_2005 force field was conducted. High throughput docking was performed and ligand poses scored using GlideScore scoring functions. The binding of top candidate compounds discovered through virtual screening against human CD24 were subsequently functionally assayed. FIG. 1D demonstrates the anti-steatosis efficacy of 3 small molecules (Compounds A, B, and C) that were identified in this way. This finding demonstrates that any agent the binds to CD24 or reduces CD24 expression (e.g., anti-CD24 CRISPR agents such as CRISPR nuclease or CRISPRi, anti-CD24 RNAi agents such as shRNA or siRNA, anti-CD24 oligonucleotide agents such as LNAs, and the like) can be used in the methods described herein (e.g., as an anti-steatosis agent).

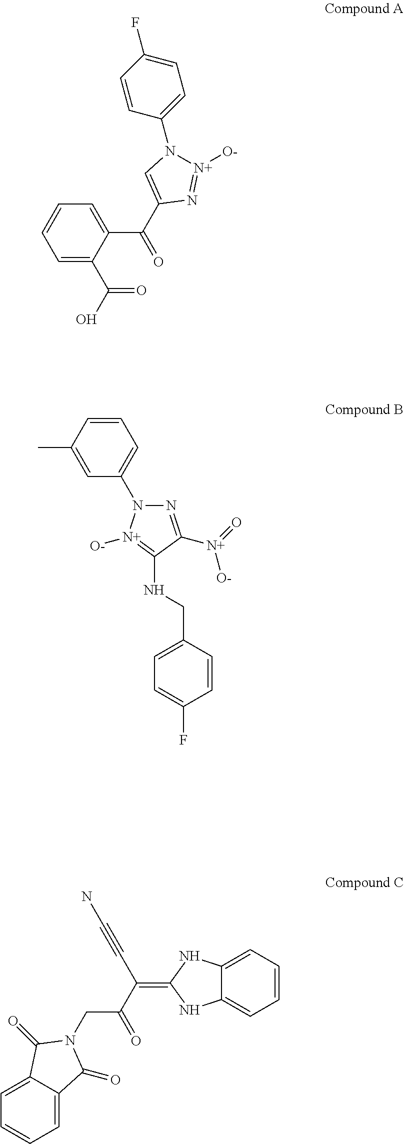 [SMILES: O=C(O)c1ccccc1C(=O)c1cn(-c2ccc(F)cc2)[n+]([O-])n1 ; Cc1cccc(-n2nc([N+](=O)[O-])c(NCc3ccc(F)cc3)[n+]2[O-])c1 ; NC#CC(C(=O)CN1C(=O)c2ccccc2C1=O)=C1Nc2ccccc2N1]

We then sought to determine the therapeutic effects of targeting CD24 against fatty liver disease in vivo.

### Administration of Anti-CD24 Antibody Inhibits Hepatic Steatosis

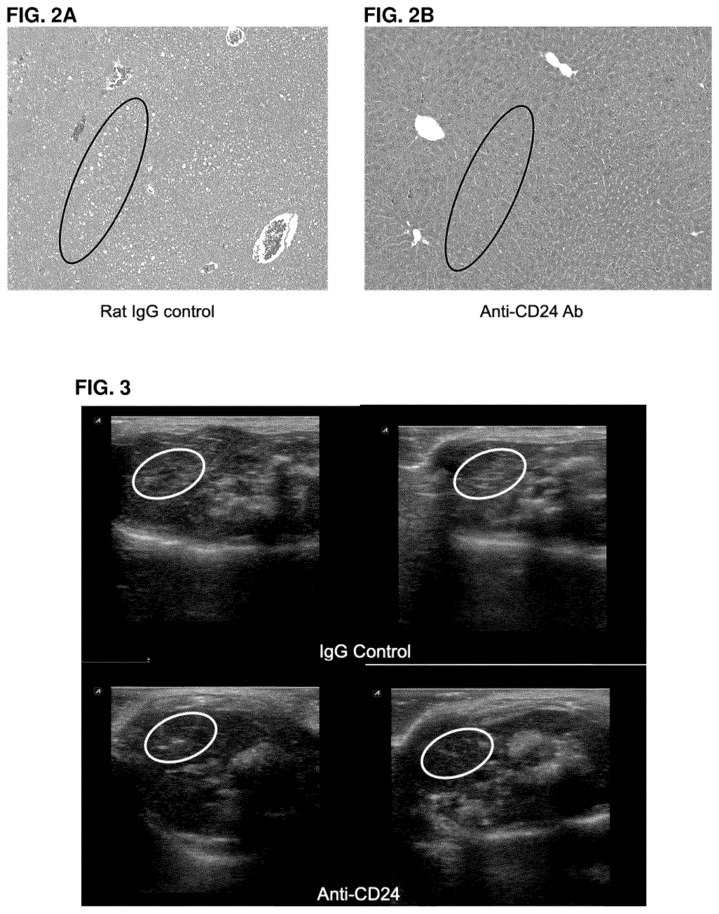

6-8 week old Rag-deficient (deficient in T cells and B cells) mice dosed with 25 μg Rat IgG2b isotype control or rat anti-mouse CD24 antibody 2×/week for 3-4 weeks subcutaneously. Mice were put on high fat diet (60% kcal from fat) 1 day after the first dose. Liver was collected after 4 weeks and liver histology in the form of hematoxylin and eosin (H&E) stain was obtained. All mice tolerated the antibodies well. As shown in representative liver histology slides in FIG. 2, mice on high fat diet developed significant hepatic steatosis (A) while mice on high fat diet treated with anti-CD24 Ab showed essentially little to no hepatic steatosis.

In addition to histology improvement, the improvement in hepatic steatosis can be monitored via ultrasound. As shown from representative images in FIG. 3, liver ultrasound of mice on HFD treated with control IgG (top) showed increased echogenicity (aka hyperechoic or that the liver appears more white) compared to mice treated with anti-CD24 agent (bottom).

### Administration of Anti-CD24 Antibody Reverses Hepatic Steatosis in 1 Week

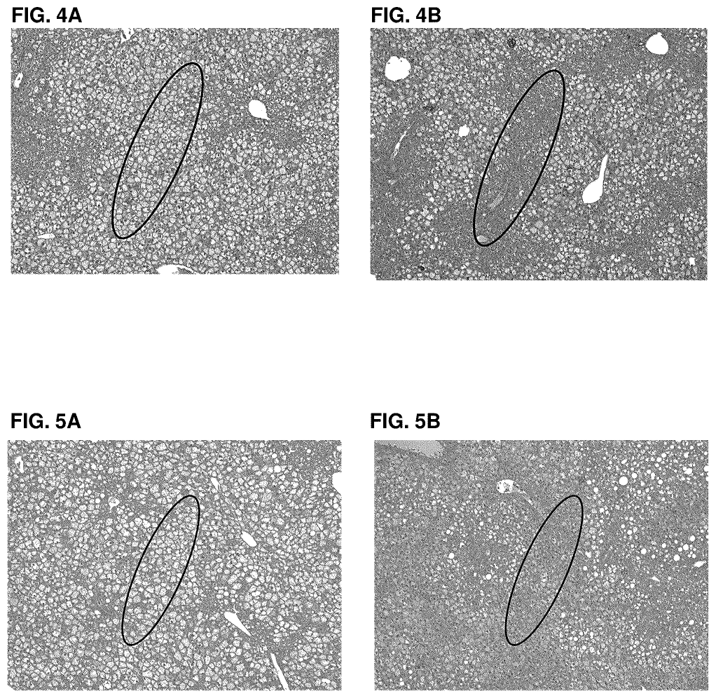

6-8-week old ob/ob mice were put on HFD (60% kcal from fat) for 1 week to accelerate the development of steatosis. They were then kept on HFD and dosed with 50 μg IgG control or anti-CD24 antibody daily for 6 days subcutaneously. Excitingly, as shown in representative histology images in FIG. 4, mice on high fat diet developed profound hepatic steatosis (A) while mice on high fat diet treated with anti-CD24 Ab for just 1 week showed significantly more normal liver architecture and significantly less steatosis (B).

Administration of Anti-CD24 Antibody can More Effectively Reverse Hepatic Steatosis when Combined with Dietary Modification.

While ob/ob mice on HFD treated with anti-CD24 agent for 1 week show significantly less steatosis compared to control treated mice, there was still appreciable steatosis. Alternatively, we hypothesize that combination treatment with anti-CD24 agent and dietary changes (i.e putting the ob/ob mice back on regular chow (4% kcal from fat) while treating them with anti-CD24 agents can more effectively reverse hepatic steatosis. To test this hypothesis, after on HFD for 1 week, ob/ob mice were placed back on regular chow diet and either treated with IgG controls or anti-CD24 agent for 6 days. Indeed, as shown in representative histology images in FIG. 5, mice treated with IgG controls still showed pronounced steatosis (A), while mice treated with anti-CD24 agents showed little steatosis.

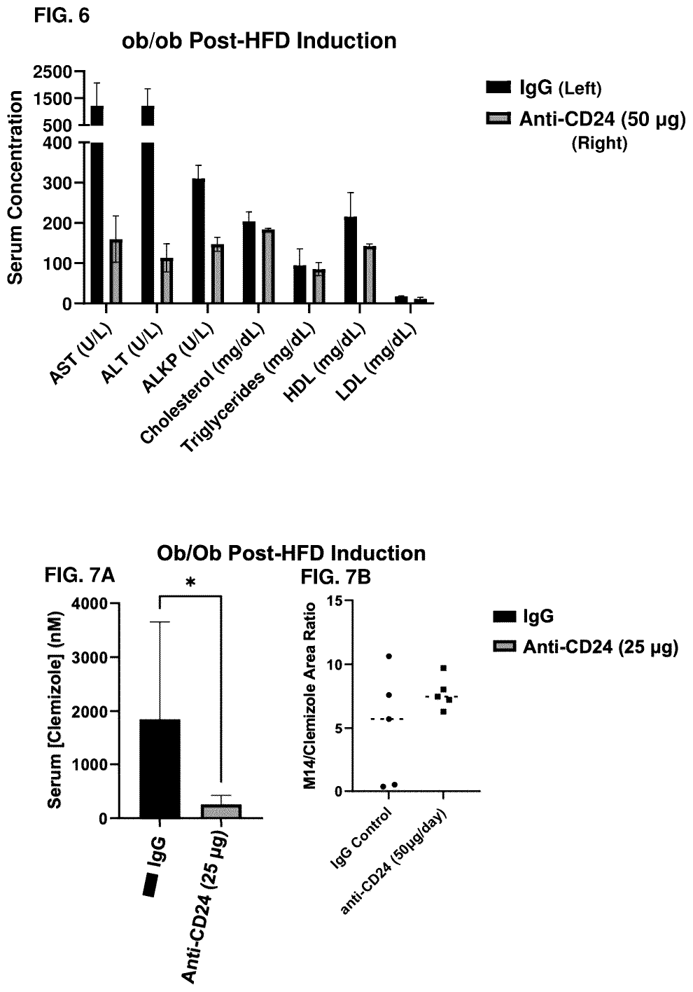

In addition to the histologic improvement in steatosis, administration of anti-CD24 antibody leads to improvement in serum liver chemistry as demonstrated by decreased serum ALT, AST, Alphos, GGT, cholesterol (FIG. 6), and was well-tolerated.

Administration of Anti-CD24 Antibody Improves Liver Function of Mice with Fatty Liver

Having demonstrated that anti-CD24 agent can show improvement in liver histology of mice that develop fatty liver disease, and improve serum liver chemistries, we wanted to determine if anti-CD24 agent can show improvement in liver function. One such function of the liver is drug metabolism. One known drug that is extensively metabolized by the liver is clemizole. We hypothesized that severely steatotic liver may have impairment in drug metabolism compared to normal liver, and that anti-CD24 agent can restore liver function in addition to improving liver histology and serum chemistry. To test this hypothesis, ob/ob mice were put on HFD (60% kcal from fat) for 1 week. 1 week after fat induction, mice were dosed daily with 25 μg/day of IgG control or anti-CD24 subcutaneously while still on HFD. 8 days after first dose of antibody, mice were dosed PO with 25 mg/kg clemizole-HCl (vehicle: water). 30 minutes after dosing, mice were euthanized, serum was collected, and the serum concentrations of clemizole and the major mouse metabolite M14)¹ were determined via quantitative mass spectrometry. Indeed, as shown in FIG. 7, ob/ob mice on HFD treated with IgG showed impairment in hepatic metabolism as shown by much higher level of serum clemizole in the IgG treated mice vs anti-CD24 treated (A). This is corroborated by a concurrent increase in serum concentrations of the major mouse metabolite (M14) in mice treated with anti-CD24 vs. IgG control (B). Taken together, this demonstrates improvement in hepatic function with anti-CD24 treatment.

### Administration of Anti-CD24 Antibody Whose Fc is Silenced for ADCC Activity Still Effectively Inhibits Steatosis

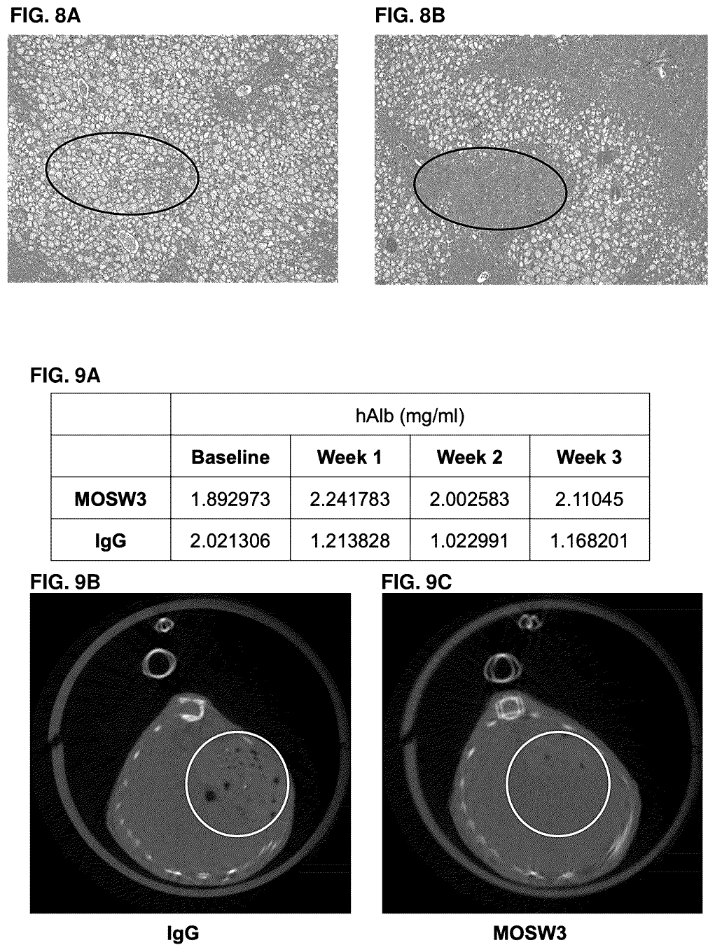

Since some antibodies may exert unwanted toxicity due to antibody-dependent cytotoxicity (ADCC) or antibody-dependent cellular phagocytosis (ADCP), we sought to determine if anti-CD24 antibodies whose Fc are devoid of ADCC and/or ADCP, and therefore have decreased toxicity, can still retain anti-steatosis activity. To test this hypothesis, we utilized an anti-CD24 antibody whose Fc domain has been engineered to contain point mutations that abrogate binding to Fc gamma receptors. These mutations result in silent ADCC activity (Absolute antibody). ob/ob mice were put on HFD for 1 week then dosed with either IgG or ADCC-silent anti-CD24 antibody (Anti-CD24 [YBM 5.10.4], Mouse IgG2a, Fc Silent Kappa; product code: Ab00210-2.3 from absolute antibody) while on regular diet for 1 week, and then livers were taken for histology. Indeed, as shown in representative histology images in FIG. 8, mice treated with IgG controls still showed pronounced steatosis (A), while mice treated with ADCC-silent anti-CD24 antibody showed much less steatosis.

Administration of Anti-CD24 Agent Improves Human Liver Engraftment, Maintains Human Albumin and Decreases Steatosis in Mice with Humanized Liver

In order to determine the effect of anti-CD24 agent in a more physiologic model of human fatty liver disease, we used a mouse model in which human liver can be engrafted. Briefly, NSG mice with humanized livers were prepared using the RAAPID-TKG method. These mice contain chimeric livers that harbor both murine and human hepatocytes and the degree of human liver engraftment can be ascertained by measuring serum human albumin. 2 mice with similar degrees of human liver engraftment, as measured by baseline serum human albumin, were placed on HFD for 6 weeks and then treated with either IgG control or an anti-CD24 antibody—which we term MOSW3—whose Fc has been engineered to contain point mutations that abrogate ADCC, ADCP, as well as compliment binding activity, i.e., Fc-silent antibody. Antibodies were dosed at 25 μg twice a week. Excitingly, as shown in FIG. 9A, treating with MOSW3 shows longer graft survival as evidenced by the maintenance of serum human albumin while IgG control treated mice showed progressive loss of human liver engraftment as shown by a steady decrease in serum human albumin. This indicates that treating with anti-CD24 agent improves the synthetic function of the liver, i.e., albumin production At 3 weeks post treatment, CT scan was also performed, where, in contrast to ultrasound, fat shows up as black areas. The IgG treated mouse showed higher level of steatosis (FIG. 9B) compared to the MOSW3 treated mouse (FIG. 9C).

This result indicates that therapeutic administration of anti-CD24 agent can decrease steatosis and increase human liver graft survival. Anti-CD24 agent can be used to treat a fatty liver before it is transplanted, or after it is in a liver transplant recipient.

Administration of Anti-CD24 Antibody can Slow the Rate of Weight Gain while on High Fat Diet.

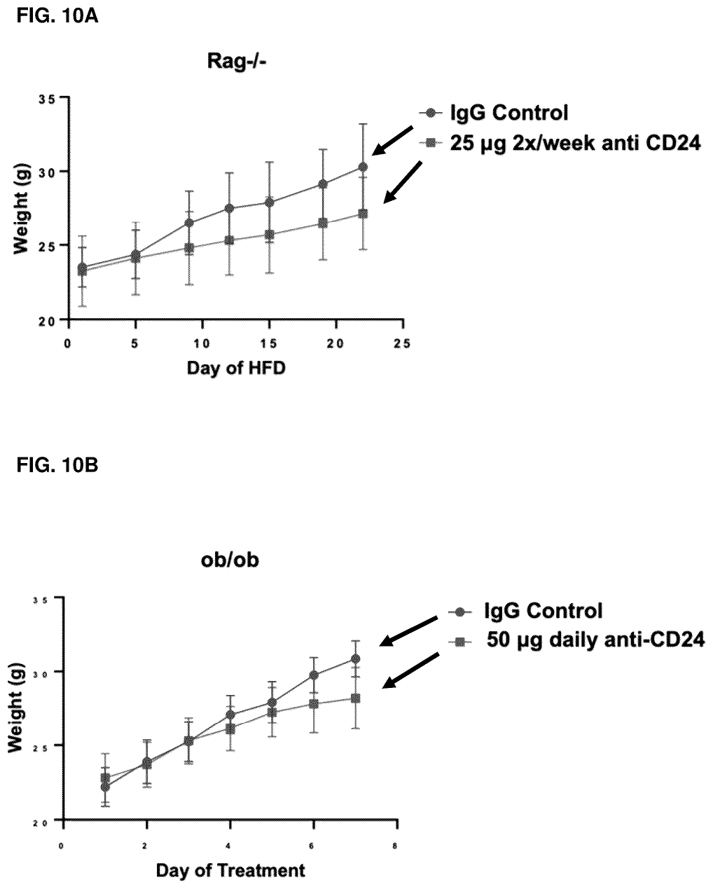

While all mice treated with anti-CD24 agents at the doses showing therapeutic benefits against fatty liver disease show no signs of weight loss or other toxicity, we observed that in some instances, therapeutic administration of anti-CD24 agent can slow the rate of weight gain in mice on HFD and this was observed in both Rag−/− and ob/ob-mice. This suggests that anti-CD24 agents can have therapeutic benefit in other obesity-associated diseases. (See FIG. 10).

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112(f) or 35 U.S.C. § 112(6) is not invoked.

The invention claimed is:

1. A method of reducing steatosis in an individual, the method comprising: administering to an individual who has steatosis, a therapeutically effective dose of an anti-CD24 agent for a period of time sufficient to reduce the steatosis.

2. The method of claim 1, wherein the subject has non-alcoholic fatty liver disease (NAFLD) or non-alcoholic steatohepatitis (NASH).

3. The method of claim 1, wherein the anti-CD24 agent is an anti-CD24 antibody.

4. The method of claim 3, wherein said anti-CD24 antibody comprises an Fc with one or more silencing mutations.

5. The method of claim 3, wherein said anti-CD24 antibody is an anti-CD24 fragment antigen-binding region (Fab) or an anti-CD24 pegylated Fab fragment.

6. The method of claim 3, wherein said anti-CD24 antibody is an anti-CD24 single-chain variable fragment (scFv).

7. The method of claim 3, wherein the anti-CD24 antibody comprises the 3 heavy chain and 3 light chain CDRs of SWA11, ML5, SN3, or G7s.

8. The method of claim 3, wherein the anti-CD24 antibody is a humanized antibody.

9. The method of claim 3, wherein the anti-CD24 antibody comprises an amino acid sequence having the 3 heavy chain IMGT CDRs of SWA11 and having 80% or more sequence identity with the humanized variable heavy chain sequence of any one of SEQ ID NOs: 2-14; and an amino acid sequence having the 3 light chain IMGT CDRs of SWA11 and having 80% or more sequence identity with the humanized variable light chain sequence of any one of SEQ ID NOs: 19-31.

10. The method of claim 1, wherein the anti-CD24 agent is a small molecule that binds to CD24.

11. The method of claim 1, wherein the course of treatment is from 1-8 weeks.

12. The method of claim 1, wherein the said administering is combined with dietary modification and/or weight loss surgery.

13. The method of claim 1, wherein the anti-CD24 agent is co-administered with another therapeutic agent.

14. The method of claim 1, wherein the individual is a liver transplant patient and said administering is to enhance liver graft function and/or prolong liver graft survival.

15. The method of claim 1, wherein the individual has fatty liver, a fatty liver associated disease, obesity, or an obesity-related disease.
The above claim amendments have been made solely to reduce the requisite filing fee. As such, the above amendments have not been made for reasons of patentability, i.e., in order to comply with any statutory requirements for patentability.
